(19) United States
(12) Patent Application Publication
(10) Pub. No.: US 2022/0233412 A1
(43) Pub. Date: Jul. 28, 2022

(54) MICROPARTICLES COMPRISING CELLULOSE NANOCRYSTALS AGGREGATED WITH PROTEINS AND COSMETIC USES THEREOF

(71) Applicant: ANOMERA INC., Montreal, Québec (CA)

(72) Inventors: Mark P. ANDREWS, Westmount , Québec (CA); Timothy MORSE, Toronto, Ontario (CA); Monika RAK, Montreal, Québec (CA); Junqi WU, Montreal, Québec (CA); Zhen HU, Mississauga, Ontario (CA); Mary BATEMAN, St. Lazarre, Quebec (CA)

(73) Assignee: ANOMERA INC., Montreal, Québec (CA)

(21) Appl. No.: 17/609,958

(22) PCT Filed: May 6, 2020
(86) PCT No.: PCT/CA2020/050603

§ 371 Date: Nov. 9, 2021

Related U.S. Application Data
(60) Provisional application No. 62/846,281, filed on May 10, 2019.

(51) Int. Cl. A61K 8/02; A61K 8/64; A61K 8/73; A61Q 19/00; A61K 8/06

(52) U.S. Cl. CPC A61K 8/0287; A61K 8/64; A61K 8/731; A61Q 19/00; A61K 2800/10; A61K 8/064; A61K 2800/412; A61K 2800/413; A61K 8/0279

(57) ABSTRACT

Proteinaceous cellulose microparticles are provided. These microparticles comprise cellulose nanocrystals and one or more peptide, one or more protein, or a mixture thereof, wherein the nanocrystals and the peptide(s) and/or protein(s) are aggregated together to form the microparticles. In embodiments, the microparticles comprise silk fibroin and advantageously are hydrophobic and lipophilic. There are also provided cosmetic preparations comprising these microparticles. In advantageous embodiments, these cosmetic preparations comprise a water-in-oil emulsion or a lipophilic medium. Finally, there are also provided methods of producing the microparticles.

### CROSS REFERENCE TO RELATED APPLICATIONS

This application claims benefit, under 35 U.S.C. § 119(e), of U.S. provisional application Ser. No. 62/846,281, filed on May 10, 2019.

### FIELD OF THE INVENTION

The present invention relates to proteinaceous cellulose microparticles. More specifically, the present invention is concerned with microparticles containing protein(s) and cellulose nanocrystals, and which are hydrophobic, have increased oil uptake, and/or improved skin feel.

### BACKGROUND OF THE INVENTION

### On Microbeads

Microparticles play important roles in drug delivery, cosmetics and skin care, in fluorescent immunoassay, as micro-carriers in biotechnology, as viscosity modifiers, stationary phases in chromatography, and as abrasives. In these fields, as well as others, microparticles are often referred to as “microbeads”.

The cosmetics and personal care industry utilize microbeads to enhance sensory properties in formulations and to provide protection to, or amelioration of, the skin. In cosmetics and skin care, microbeads are used to impart a variety of consumer recognized benefits such as, but not limited to: thickening agent, filler, volumizer, color dispersant, exfoliant, improved product blending, improved skin feel, dermatological benefits, soft focusing (also known as blurring), product slip, oil uptake, and dry binding. Soft focus or blurring is a property of microbeads due to their ability to scatter light. Oil uptake refers to the capacity of the microbead to absorb sebum form the skin. This property allows cosmetic formulators to design products that impart a mattifying effect to make-up so that a more natural look extends over periods of hours of wear.

Generally speaking, microbeads can be produced from plastics, glass, metal oxides and naturally occurring polymers, like proteins and polysaccharides including starches and cellulose. In the cosmetics industry, microbeads are conventionally made from plastics.

There is compelling evidence that microbeads made from plastics cause harm to the environment, including damage along the food chain. Increased consumer concern regarding personal health and environmental health has stimulated growth in organic/natural personal care products. Effective organic/natural replacements for traditional products along with societal lifestyle changes are important motivators for widespread adoption not only of “green” personal care products, but also of sustainable ingredients for inks, pigments, coatings, composites and thickeners for paints. Regarding sustainability, it is desirable to use “green chemistry” and “green engineering” methods that use renewable resources to make microbeads. The use of “green chemistry” and “green engineering” methods that use renewable resources to make microbeads that are designed to degrade is known to have a positive impact on sustainability.

In the cosmetics industry, it is not straightforward to replace plastic microbeads with microbeads made solely from proteins, cellulose, chitosan, starch or silica. This is because the mechanical, optical and surface properties of these materials differ from those of plastics. The cosmetics industry has invested significant capital, know-how and research into plastic microbeads to build markets for cosmetics and skin care. The pressure to replace plastic microbeads with environmentally friendly alternatives means that the cosmetics industry must align its formulations and products with the properties of the replacement particles.

Plastic microbeads are generally hydrophobic/lipophilic. This makes them advantageous for use in hydrophobic or lipophilic formulations. However, in some cases, it is desirable to use plastic microbeads that are hydrophilic. Plastic microbeads may be made hydrophilic by coating their surface with compounds like carboxylate, sulfate, sulfonate, quaternary ammonium, alcohol, amino or amide groups that make hydrogen bonds with polar host fluids.

On the other hand, microbeads made from proteins, starches, cellulose, chitosan, and silica are generally hydrophilic. Most generally, these types of microbeads must be coated to make them hydrophobic/lipophilic so that they are compatible with hydrophilic/lipophilic host media like oils, waxes and many petroleum-based solvents and can therefore replace the ubiquitous hydrophobic/lipophilic plastic microbeads.

### On Lipophilic Microbeads

Lipophilicity may be expressed as log P which describes the partitioning of the neutral molecules between the two matrices. Lipophilicity may also be expressed as log D which describes the partitioning of the neutral fraction of the molecule population plus the partitioning of the ionized fraction of the molecule population between the two matrices. Lipophilicity (expressed as log P) is a molecular parameter encoding both electrostatic and hydrophobic intermolecular forces as well as intramolecular interactions.

The terms “lipophilic” and “hydrophobic” are not synonymous, as can be seen with silicones and fluorocarbons, which are hydrophobic but not lipophilic. The International Union of Pure and Applied Chemistry (IUPAC) provides different definitions for lipophilicity and hydrophobicity (Van de Waterbeem, H.; Carter, R. E.; Grassy, G.; Kubinyi, H.; Martin, Y. C.; Tute, M. S.; Willett, P. Pure Appl. Chem. 1997, 69, 1137-1152.). Hydrophobicity is the association of nonpolar groups or molecules in an aqueous environment which arises from the tendency of water to exclude nonpolar molecules. Lipophilicity represents the affinity of a molecule or a moiety for a lipophilic environment.

There is a need in the cosmetic industry for lipophilic microbead texturizing agents.

Generally, the surfaces of microbeads must be modified in order to make them compatible with cosmetic formulation. To aid cosmetic formulation, provide functional properties and enhance the aesthetic experience, microbeads of cellulose, starch and silica are usually subjected to various kinds of surface treatments. These treatments alter the surface energy of the microbeads in ways that improve formulation and the sensorial experience.

Lauroyl lysine is one example of a surface treatment agent that creates a hydrophobic surface that favors enhanced particle dispersion, improved wear properties and make-up with a wet feel on the skin.

Alkylsilane coatings result from the reaction of organosilicon alkoxides with surface water and hydroxyl groups on cellulose, starch or silica particles. Covalent bonds are formed among the silicon moieties and with the particle surface following curing.

Silicone treated particles disperse well in cyclomethicones. They have very low surface tension, giving them excellent hydrophobicity and improved lipophilicity. The coating makes the particles easily dispersible in mineral oils, esters and silicone fluids. Particles treated with alkyl silane are more hydrophobic than methicone treated particles, wet better in commonly utilized cosmetic oils and have lower oil absorption.

In hydrous compact formulations, alkyl silane treatment imparts improved wetting to allow high particle loading in powders. This confers a ‘powdery’ sensation upon application to the skin while maintaining a low melt viscosity for hot filling. The improvement in compatibility between the dispersed solids and the vehicle is a benefit in formulation of stick products including lipstick, eye shadows and foundations. These types of coatings are used to make W/O (water-in-oil) and O/W (oil-in-water) emulsions, water-proof mascara, long lasting lipstick and lip gloss.

Methicone is a poly(methylhydrosiloxane). The Si—H bond reacts with traces of water from a particle surface and converts the Si—H bond to silanol (Si—OH), which ultimately condenses to make covalent Si—O particle chemical bonds. The coating is highly hydrophobic and is tenaciously bound to the surface so that the coating resists shear. Particles coated this way wet well in oils, particularly silicone oils. The skin feel is experienced as somewhat dry with enhanced slip and spreadability. It is preferred for pressed powder formulations. A drawback of the coating is that the methicone reaction must be taken to completion since the reaction evolves hydrogen gas. Methicone coated particles are suitable for foundations, concealers, mascaras, lipsticks, eye shadow, and mousses.

Dimethicone is the polymer, poly(dimethylsiloxane). It is thought to bind to a particle surface via the mechanism of hydrolysis, condensation and curing to create a Si—O particle linkage. Surfaces treated with dimethicone are quite hydrophobic and have good slip and more lubricious feel. Particles coated with dimethicone are useful in oil-based systems, which may be used for anhydrous products.

The coating methods described above require the addition of several steps after production of the particle.

### On Proteinaceous Microbeads

In the cosmetics industry, there is demand for amino acid, peptide and/or protein-containing microbeads. Microbeads made from these proteins, even when blended with other polymers to try to improve stability, have the negative feature in that they have poor mechanical properties and they have a high degradation rate. For example, some microparticles of starch blends with silk fibroin dissolve up to nearly 65% when placed in water (Y. Baimarck et al., “Morphology and thermal stability of silk fibroin/starch blended microparticles”, Polymer Letters Vol. 4, No. 12 (2010) 781-789; DOI: 10.3144/expresspolymlett.2010.94). This is undesirable when formulating microbeads in emulsions containing water under conditions of shear mixing, or in formulations with high water content.

Prior art concerned with protein-based microbeads focuses on the use of gelatin, silk fibroin, sericin, and collagen. Gelatin is a biodegradable natural protein polymer that can be used to produce microparticles. However, due to the aqueous solubility and limited mechanical and thermal properties of gelatin microparticles, improvements, such as chemical crosslinking reactions, are necessary in order to provide use in long term applications. Silk fibroin, sericin, and collagen absorb water, a property that makes them unsuitable for an important class of cosmetic formulations called water-in-oil emulsions.

### On Cellulose and Cellulose Microbeads

Natural cellulose is a hydrophilic semi-crystalline organic polymer. It is a polysaccharide that is produced naturally in the biosphere. It is the structural material of the cell wall of plants, many algae, and fungus-like oomycota. Cellulose is naturally organized into long linear chains of ether-linked poly(β-1,4-glucopyranose) units. These chains assemble by intra- and inter-molecular hydrogen bonds into highly crystalline domains of nanocrystals—see FIG. 1. Regions of disordered (amorphous) cellulose exist between these nanocrystalline domains in the cellulose nanofibrils. Extensive hydrogen bonding among the cellulose polymer chains makes cellulose extremely resistant to dissolution in water and most organic solvents, and even many types of acids.

Cellulose is widely used as a nontoxic excipient in food and pharmaceutical applications. In medical applications like oral drug delivery, drugs are mixed with cellulose powder (usually microcrystalline cellulose powder) and other fillers and converted by extrusion and spheronisation. Extrusion and spheronisation yield granulate powders. Porous microbeads can be used to make a chromatographic support stationary phase for size exclusion chromatography and as selective adsorbents for biological substances such as proteins, endotoxins, and viruses.

International patent publication no. WO 2016\015148 A1, incorporated herein by reference, teaches how to produce nanocrystals of crystalline nanocellulose and then to aggregate these nanocrystals into roughly spherical (globular) microbeads by spray-drying.

### SUMMARY OF THE INVENTION

In accordance with the present invention, there is provided:
1. A proteinaceous cellulose microparticles comprising cellulose nanocrystals and one or more peptide, one or more protein, or a mixture thereof, wherein the nanocrystals and the peptide(s) and/or protein(s) are aggregated together to form the microparticles
2. The microparticles of item 1, wherein the microparticles are from about 1 μm to about 100 μm in diameter.
3. The microparticles of item 1 or 2, wherein the microparticles have a size distribution (D₁₀/D₉₀) of about 5/15 μm to about 5/25 μm by volume.
4. The microparticles of any one of items 1 to 3, wherein the microparticles are roughly spheroidal or hemi-spheroidal.
5. The microparticles of any one of items 1 to 4, wherein the cellulose nanocrystals are from about 50 nm to about 500 nm in length and from about 2 to about 20 nm in width.
6. The microparticles of any one of items 1 to 5, wherein the cellulose nanocrystals have a crystallinity of at least about 50%.
7. The microparticles of any one of items 1 to 6, wherein the cellulose nanocrystals are sulfated cellulose nanocrystals and salts thereof, carboxylated cellulose nanocrystals and salts thereof, and their derivatives such as surface-reduced carboxylated cellulose nanocrystals and salts thereof, as well as cellulose nanocrystals chemically modified with other functional groups, or a combination thereof.
8. The microparticles of any one of items 1 to 7, wherein the cellulose nanocrystals are carboxylated cellulose nanocrystals and salts thereof, preferably carboxylated cellulose nanocrystals or cellulose sodium carboxylate salt, and more preferably carboxylated cellulose nanocrystals.
9. The microparticles of any one of items 1 to 8, wherein the peptide and the protein are water-soluble.
10. The microparticles of any one of items 1 to 9, wherein the microparticles of the invention comprise one or more protein.
11. The microparticles of any one of items 1 to 10, wherein the microparticles comprise silk fibroin, sericin, or gelatin, preferably sericin or silk fibroin, and more preferably silk fibroin.
12. The microparticles of item 11, comprise silk fibroin.
13. The microparticles of any one of items 1 to 12, being hydrophobic and lipophilic
14. The microparticles of any one of items 1 to 13, wherein the microparticles comprise the one or more peptide and/or the one or more protein in a total peptide and protein concentration of about 0.1 wt % to about 50 wt %, preferably from about 0.5 wt % to about 20 wt %, and more preferably about 1 wt % to about 20 wt %, based on the weight on the microparticle
15. The microparticles of any one of items 1 to 14, wherein the microparticles are porous and the nanocrystals and the peptide and/or protein are arranged around cavities in the microparticles, thus defining pores in the microparticles.
16. The microparticles of any one of items 1 to 15, wherein the pores in the microparticles are from about 10 nm to about 2000 nm in size, preferably from about 50 to about 100 nm in size
17. The microparticles of any one of items 1 to 16, wherein the microparticles further comprise one or more functional molecules that bring additional benefits to the skin, such as protection against ultraviolet light and blue light, antioxidant properties, anti-aging properties, moisturizing effects, or color.
18. The microparticles of any one of items 1 to 17, wherein the cellulose nanocrystals are coated with a polyelectrolyte layer and a dye.
19. A cosmetic preparation comprising the microparticles of any one of items 1 to 18.
20. The cosmetic preparation of item 19, being comprising a water-in-oil emulsion or a lipophilic medium.
21. A method for producing the microparticles of any one of items 1 to 18, the method comprising the steps of:
   a) providing a suspension of cellulose nanocrystals and a solution of the one or more peptide, one or more protein, or mixture thereof;
   b) mixing the suspension with the solution to produce a mixture; and
   c) spray-drying the mixture to produce the microparticles.
22. The method of item 21, wherein the solution contains the one or more peptide, the one or more protein, or the mixture thereof in a concentration from about 0.01 wt % to about 50 wt % based on the total weight of the solution.
23. The method of item 21 or 22, further comprising the step of washing the microparticles with an alcohol.
24. The method of any one of items 21 to 23, wherein:
   after step b) a functional molecule is dissolved or suspended in the mixture of step b);
   during step a) a functional molecule is dissolved or suspended in the suspension of cellulose nanocrystals; or
   during step a) a functional molecule is dissolved or suspended in the solution of the one or more peptide, one or more protein, or mixture thereof.
25. A method for producing the microparticles of any one of items 1 to 16 that are porous, the method comprising the steps of:
   a) providing:
      a suspension of cellulose nanocrystals,
      a solution of the one or more peptide, one or more protein, or mixture thereof, and
      an emulsion of a porogen,
   wherein the solution of the one or more peptide, one or more protein, or mixture thereof either is part of the emulsion or stands alone;
   b) mixing the suspension with the solution and the emulsion to produce a mixture comprising a continuous liquid phase in which:
      droplets of the porogen are dispersed,
      the cellulose nanocrystals are suspended and
      the one or more peptide, one or more protein, or mixture thereof is dissolved;
   c) spray-drying the mixture to produce microparticles; and
   d) if the porogen has not sufficiently evaporated during spray-drying to form pores in the microparticles, evaporating the porogen or leaching the porogen out of the microparticles.

### BRIEF DESCRIPTION OF THE DRAWINGS

In the appended drawings:

FIG. 1 is a schematic representation of cellulose fibers, fibrils, nanofibrils (CNF), and nanocrystals (CNC).

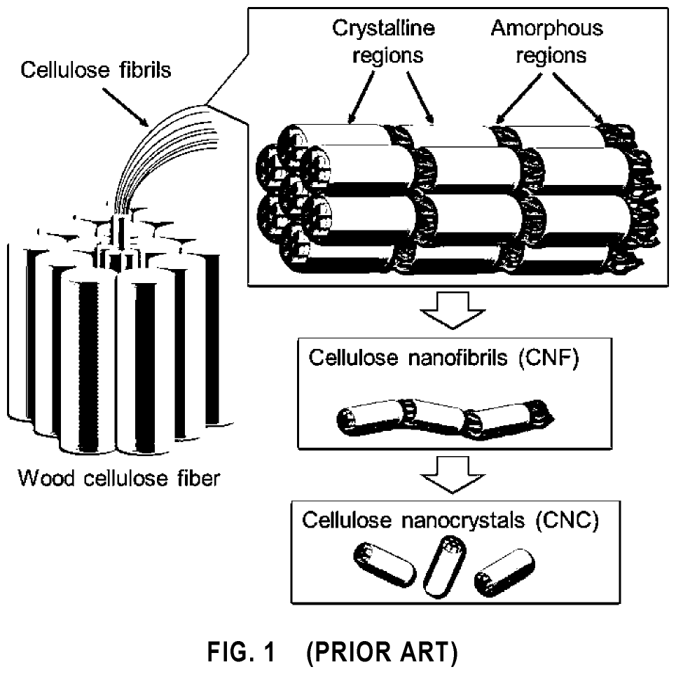

FIG. 2a) shows the powder obtained in Example 1 to which water was added—the powder sits on the surface of the water droplet, rather than being wetted.

FIG. 2b) shows the powder obtained in Comparative Example 8 to which water was added—the powder was wetted.

FIG. 3a) shows the powder obtained in Example 1 mixed in a water-in-oil emulsion—no aggregates can be observed.

FIG. 3b) shows the powder obtained in Comparative Example 8 mixed in a water-in-oil emulsion—aggregates are visible.

FIG. 4a) is a scanning electron micrograph (SEM) image of microparticles of Example 2 containing 2% silk fibroin.

FIG. 4b) is a SEM image of microparticles of Example 2 containing κ% silk fibroin.

FIG. 4c) is a SEM image of microparticles of Example 2 containing 10% silk fibroin.

FIG. 4d) is a SEM image of microparticles of Example 2 containing 20% silk fibroin.

FIG. 5 is a SEM image of microparticles consisting of 100% silk fibroin.

FIG. 6 shows the percentage of beta-pleated sheet in 2% silk fibroin/CNC microbeads before and after exposure of the microparticles to methanol. The percent contribution was obtained by Gaussian deconvolution infrared spectrum of the amide stretching region of the sample.

FIG. 7 shows the x-ray photoelectron spectrum of a hybrid microparticle containing 2% silk fibroin.

FIG. 8 shows the methylene blue dye uptake of a hybrid CNC microparticle containing 2% silk fibroin (a) as prepared and (b) after exposure of the microbead to methanol.

### DETAILED DESCRIPTION OF THE INVENTION

Turning now to the invention in more details, there is provided proteinaceous cellulose microparticles, their method of making and their use.

Indeed, it has been surprisingly found that the incorporation of one or more peptide, one or more protein, or a mixture thereof in cellulose microparticles by aggregating together of the protein and cellulose nanocrystals (CNCs) conferred surprising properties to the microparticles. In particular, the microparticles can be made hydrophobic, their oil uptake can be increased, and/or their skin feel can be improved.

Thus, the microparticles of the invention comprise cellulose nanocrystals and one or more peptide, one or more protein, or a mixture thereof, wherein the nanocrystals and the peptide(s) and/or protein(s) are aggregated together to form the microparticles.

In the microparticle of the invention, the nanocrystals are aggregated together to form the microparticles. This means that the physical structure of the microparticles is provided by the agglomerated nanocrystals.

In embodiments, the microparticles are typically free from each other, but some of them may be peripherally fused with other microparticles.

In embodiments, the microparticles are in the form of a free-flowing powder.

In embodiments, the microparticles are from about 1 μm to about 100 μm in diameter, preferably about 1 μm to about 25 μm, more preferably about 2 μm to about 20 μm, and yet more preferably about 4 μm to about 10 μm. For cosmetic application, preferred sizes are about 1 μm to about 25 μm, preferably about 2 μm to about 20 μm, and more preferably about 4 μm to about 10 μm.

In embodiments, the microparticles have a size distribution (D₁₀/D₉₀) of about 5/15 μm to about 5/25 μm by volume.

In embodiments, the microparticles are roughly spheroidal or hemi-spheroidal. Herein, a “spheroid” is the shape obtained by rotating an ellipse about one of its principal axes. Spheroids include spheres (obtained when the ellipse is a circle). Herein, a “hemispheroid” is about one half of a spheroid. The deviation from the shape of a sphere can be determined by an instrument that performs image analysis, such as a Sysmex FPIA-3000. Sphericity is the measure of how closely the shape of an object approaches that of a mathematically perfect sphere. The sphericity, t-P, of a particle is the ratio of the surface area of a sphere (with the same volume as the particle) to the surface area of the particle. It can be calculated using the following formula:

$$Ψ = \frac{{π}^{1 / 3} - {( 6 {V}_{p} )}^{2 / 3}}{{A}_{p}}$$

wherein Vₚ is the volume of the particle and Aₚ is the surface area of the particle. In embodiments, the sphericity, t-P, of the microparticles of the invention is about 0.85 or more, preferably about 0.9 or more and more preferably about 0.95 or more.

### The Cellulose Nanocrystals

As noted above, the microparticles of the invention comprise cellulose nanocrystals.

In embodiment, the cellulose nanocrystals are from about 50 nm to about 500 nm, preferably from about 80 nm to about 250 nm, more preferably from about 100 nm to about 250 nm, and yet more preferably from about 100 to about 150 nm in length.

In more preferred embodiment, the cellulose nanocrystals are from about 2 to about 20 nm in width, preferably about 2 to about 10 nm and more preferably from about 5 nm to about 10 nm in width.

In embodiment, the cellulose nanocrystals have a crystallinity of at least about 50%, preferably at least about 65% or more, yet more preferably at least about 70% or more, and most preferably at least about 80%.

The cellulose nanocrystals in the microparticles of the invention may be any cellulose nanocrystals.

In particular, the nanocrystals may be functionalized, which means that their surface has been modified to attached functional groups thereon, or unfunctionalized (as they occur naturally in cellulose). The most common methods of manufacturing cellulose nanocrystals typically cause at least some functionalization of the nanocrystals surface. Hence, in embodiments, the cellulose nanocrystals are functionalized cellulose nanocrystals.

In embodiments, the cellulose nanocrystals in the microparticles of the invention are sulfated cellulose nanocrystals and salts thereof, carboxylated cellulose nanocrystals and salts thereof, and their derivatives such as surface-reduced carboxylated cellulose nanocrystals and salts thereof, as well as cellulose nanocrystals chemically modified with other functional groups, or a combination thereof.

Examples of salts of sulfated cellulose nanocrystals and carboxylated cellulose nanocrystals include the sodium salt thereof.

Examples of “other functional groups” as noted above include esters, ethers, quaternized alkyl ammonium cations, triazoles and their derivatives, olefins and vinyl compounds, oligomers, polymers, cyclodextrins, amino acids, amines, proteins, polyelectrolytes, and others. The cellulose nanocrystals chemically modified with these “other functional groups” are well-known to the skilled person. These “other functional groups” are used to impart one or more desired properties to the cellulose nanocrystals including, for example, fluorescence, compatibility with organic solvents and/or polymers for compounding, bioactivity, catalytic function, stabilization of emulsions, and many other features as known to the skilled person.

Preferably, the cellulose nanocrystals in the microparticles are carboxylated cellulose nanocrystals and salts thereof, preferably carboxylated cellulose nanocrystals or cellulose sodium carboxylate salt, and more preferably carboxylated cellulose nanocrystals.

Sulfated cellulose nanocrystals can be obtained by hydrolysis of cellulose with concentrated sulfuric acid and another acid. This method is well-known and described for example in Habibi et al. 2010, Chemical Reviews, 110, 3479-3500, incorporated herein by reference.

Carboxylated cellulose nanocrystals can produced by different methods for example, TEMPO- or periodate-mediated oxidation, oxidation with ammonium persulfate, and oxidation with hydrogen peroxide. More specifically, the well-known TEMPO oxidation can be used to oxidize cellulose nanocrystals. Carboxylated cellulose nanocrystals can be produced directly from cellulose using aqueous hydrogen peroxide as described in WO 2016/015148 A1, incorporated herein by reference. Other methods of producing carboxylated cellulose nanocrystals from cellulose include those described in WO 2011/072365 A1 and WO 2013/000074 A1, both incorporated herein by reference.

The cellulose nanocrystals modified with the “other functional groups” noted above can be produced from sulfated and/or carboxylated CNC (without dissolving the crystalline cellulose) as well-known to the skilled person.

### Peptides and Proteins

As noted above, the microparticles of the invention also comprise one or more peptide, one or more protein, or a mixture thereof.

Peptides are short chains of amino acids linked by peptide (amide) bonds. Proteins are also chains of amino acids linked by peptide bonds, but they are larger molecules comprising of one or more long chains of amino acid also linked by peptide bonds. Peptides are generally distinguished from proteins on the basis of size, and as an arbitrary benchmark can be understood to contain approximately 50 or fewer amino acids. Therefore, herein peptides are defined as comprising between 2 and 50 amino acids and proteins are defined as containing more than 50 amino acids. Enzymes constitute a subset of proteins, which are biological catalysts that accelerate chemical reactions by lowering their activation energy.

Preferably, the peptide comprises between 10 and 50 amino acids.

Preferably, the protein comprises 150 amino acids or more and therefore has a molecular weight of approximately 22 kDa or more. Most preferably, the protein is a high molecular weight polypeptide having a molecular weight of 100 kDa or more.

In preferred embodiments, the microparticles of the invention comprise one or more protein. In more preferred embodiments, the microparticle comprise one protein. In alternative embodiments, the microparticle of the invention comprise a peptide.

The peptide or protein in the microparticle of the invention can be any peptide or protein.

The peptide or protein may be natural, plant (vegetable), or animal derived peptide or protein, as well as synthetic peptide or protein and transgenic peptide or protein.

Preferred peptides and proteins include water-soluble peptides and proteins.

Non-limiting examples of peptides and proteins include albumin, amylase, amyloglucosidase, lysine polypeptide, casein, catalase, collagen, cytochrome C, deoxyribonuclease, elastin, fibronectin, gelatin, gliadin, glucose oxidase, glycoproteins, esters of hydrolyzed collagen, corn protein, keratin, lactoferrin, lactoglobulin, lactoperoxidase, lipase, milk protein, nisin, oxido reductase, papin, pepsin, protease, saccharomyces polypeptides, sericin, serum albumin, serum protein, silk fibroin, sodium stearoyl lactalbumin, soluble proteoglycan, soybean palmitate, soy protein isolate, egg protein, peanut protein, cottonseed protein, sunflower protein, pea protein, whey protein, fish protein, seafood protein, subtilisin, superoxide dismutase, sutilains, sweet almond protein, urease, wheat germ protein, wheat protein, whey protein, zein, hydrolyzed vegetable protein, and so on.

Preferred peptides and proteins in the microparticles in the invention are those that bind to cellulose without forming chemical bonds.

Preferred peptides and proteins include the following:
Glycinin and 3-conglycinin, which are the main proteins present in soy, and which adsorb onto cellulose by means of hydrogen bonding.
Glycinin is a hexamer with a molecular mass of 300-380 kDa. The six sub-units consist of acidic and basic polypeptides linked through disulfide bonds. Glycinin adsorbs onto cellulose according a Langmuir isotherm.
β-Conglycinin is a trimer or hexamer composed of two similar cysteine-containing peptides, and a glycosylated, non-cysteine-containing β peptide. β-conglycinin adsorbs onto cellulose to a lower extent.
Both proteins undergo ionic strength-dependent conformational transitions when they bind to cellulose.
Bovine serum albumin protein, which does not bind significantly to negatively charged cellulose nanocrystals bearing sulfate and/or carboxylic functionalities.
Gelatin, which is a mixture of peptides and proteins produced by partial hydrolysis of collagen.
Cellulose-degrading enzymes (cellulases), which have a specific affinity to cellulose surfaces; probably due to hydrogen bonding interactions, coupled with conformational changes to the enzyme.
Silk sericin (SS), which is a natural hydrophilic protein.
Sericin forms the gum coating around silk fibres and allowing them to adhere. Sericin is composed of 18 different amino acids, 32% of which are serine.
Silk fibroin (SF), which is a biodegradable and biocompatible natural protein polymer produced by silkworms, such as the Bombyx mori silkworm, that bond natural polysaccharides via hydrogen bond and electrostatic interactions, without forming covalent chemical bonds.
SF has a molecular mass of around 400 kDa. It is a linear polypeptide, whose main components, glycine and alanine, are non-polar amino acids. The hydrophobic domains of the so-called H-chain contain a repetitive hexapeptide sequence of Gly-Ala-Gly-Ala-Gly-Ser and repeats of Gly-Ala/Ser/Tyr dipeptides, which can form stable anti-parallel 3-sheet crystallites.
SF can exist in three molecular conformations:
   Silk I is water soluble and characterized by a mixture mainly of random coil, with some alpha-helix and beta-turn features.
   Silk II is characterized by a predominance of beta-sheet which leads to a stable and water insoluble fibroin.
   Silk III adopts an alpha-helix and is usually found at the water/air interface.
According to Feng et al. (Facile Preparation of Biocompatible Silk Fibroin/Cellulose Nanocomposite Films with High Mechanical Performance; DOI: 10.1021/acssuschemeng.7b01161; ACS Sustainable Chem. Eng. 2017, 5, 6227-6236) contact angle measurements reveal that pure SF films are hydrophilic. This property is attributed to the presence of hydrophilic hydroxyl, amino, and carboxyl groups.
SF molecules adsorb onto cellulose surfaces via either weak or strong interactions, without forming covalent chemical bonds. The resulting composites can exhibit silk I and silk II structures, or combinations of both. In many cases, films of SF are hydrophilic.

In preferred embodiments, the microparticles comprise silk fibroin, sericin, or gelatin, preferably sericin or silk fibroin, and more preferably silk fibroin. Silk fibroin allows tailoring the properties of the microparticles from hydrophilic to hydrophobic/lipophobic. On the other hand, sericin allowed producing microparticles with improved (creamier) skin feel.

The microparticles typically comprise the one or more peptide and/or the one or more protein in a total peptide and protein concentration of about 0.1 wt % to about 50 wt %, preferably from about 0.5 wt % to about 20 wt %, and more preferably about 1 wt % to about 20 wt %, based on the weight on the microparticle.

As demonstrated in the examples below, silk fibroin produced hydrophobic microparticles even when used in a concentration as low as 0.5 wt %, based on the weight on the microparticle. Thus, in embodiments, the microparticles comprise between about 0.5 wt % and about 30 wt %, preferably between about 1 wt % and about 30 wt %, and more preferably between about 2 wt % and about 30 wt %, based on the weight on the microparticle, of silk fibroin.

### Porous Microparticles

In embodiments, the microparticles of the invention are porous (i.e. they comprise pores). The nanocrystals and the peptide and/or protein are aggregated together thus forming the microparticles, and arranged around cavities in the microparticles, thus defining pores in the microparticles.

In the microparticles of the invention, cellulose nanocrystals are aggregated together forming the microparticles and defining the pores. As will be explained in the section entitled “Method for Producing the Porous Cellulose Microparticles” below, the microparticles of the invention can be produced by aggregating cellulose nanocrystals and the protein together around droplets of a porogen and then removing the porogen, thus leaving behind voids where porogen droplets used to be, i.e. thus creating pores in the microparticles. This results in nanocrystals and the one or more aggregated together and forming the microparticles themselves as well as defining (i.e. marking out the boundaries of) the pores in the microparticles.

In embodiments, the pores in the microparticles are from about 10 nm to about 2000 nm in size, preferably from about 50 to about 100 nm in size.

The porosity of microparticles can be measured by different methods. One such method is the fluid saturation method as described in the US standard ASTM D281-84. In this method, the oil uptake of a porous microparticle powder is measured. An amount p (in grams) of microparticle powder (between about 0.1 and 5 g) is placed on a glass plate or in a small vial and castor oil (or isononyl isononanoate) is added dropwise. After addition of 4 to 5 drops of oil, the oil is incorporated into the powder with a spatula. Addition of the oil is continued until a conglomerate of the oil and powder has formed. At this point the oil is added one drop at a time and the mixture is then triturated with the spatula. The addition of the oil is stopped when a smooth, firm paste is obtained. The measurement is complete when the paste can be spread on a glass plate without cracking or forming lumps. The volume Vs (expressed in ml) of oil is then noted. The oil uptake corresponds to the ratio Vs/p. In embodiments, the microporous particles of the invention have a castor oil uptake of about 60 ml/100 g or more. In preferred embodiments, the castor oil uptake is about 65, about 75, about 100, about 125, about 150, about 175, about 200, about 225, or about 250 ml/100 g or more.

The porosity of microparticles can also be measured by the BET (Brunauer-Emmett-Teller) method, which is described in the Journal of the American Chemical Society, Vol. 60, p. 309, 1938, incorporated herein by reference. The BET method conforms to the International Standard ISO 5794/1. The BET method yields a quantity called the surface area (m²/g). In embodiments, the microporous particles of the invention have a surface area of about 30 m²/g or more. In preferred embodiments, the surface area is about 45, about 50, about 75, about 100, about 125, or about 150 m²/g or more.

### Optional Components in the Microparticles

In addition to the peptide/protein, in embodiments, the microparticles of the invention can also comprise one or more functional molecules that bring additional benefits to the skin. These benefits include for example protection against ultraviolet light and blue light, antioxidant and anti-aging properties, moisturizing effects, and color.

Functional molecules imparting color include natural dyes. Non-limiting examples of natural dyes include adonirubin, astaxanthin, bixin, canthaxathin, beta-apo-4-carotenal, beta-apo-8-carotenal, beta-carotene, beta-apo-8-carotenoic esters, chlorophylcitraxanthin, cryptoxanthin, echinenone, lycopene, lutein, neurosporene, torularhodin, torulene, and zeaxanthin.

Functional molecules providing direct protection against UVB or UVA light (i.e. UV (UVB or UVA) protectors) include organic oil- or water-soluble UV protectors. Non-limiting examples of oil-soluble UVB protectors include 3-benzylidenecamphor and its derivatives, 4-aminobenzoic acid derivatives, esters of cinnamic, benzalmalonic and salicylic acid, derivatives of benzophenone and derivatives of triazines. Non-limiting examples of water-soluble UVB protectors include derivatives mainly of sulfonic acid and its salts. Examples are 2-benzyphenylimidazole-5-sulfonic acid and its salts, sulfonic acid and the salts thereof of 3-benzylidenecamphor, sulfonic acid and the salts thereof or benzophenones. Non-limiting examples UVA protectors are derivatives of benzoylmethane and aminohydroxy-substituted derivatives of benzophenone.

Some functional molecules provide secondary benefits to the above UV protectors because they exhibit antioxidant properties. Non-limiting examples of such anti-oxidant functional molecules include vitamin E, coenzyme Q10, quinones, ubiquinones, and vitamin C (ascorbic acid). These anti-oxidant functional molecules interrupt the photochemical chain reaction that occurs when UV light penetrates the skin.

Other functional molecules that are UV protectors include inorganic pigments like titanium dioxide and zinc oxide. These can be combined with molecules that provide direct protection against UVB or UVA light discussed above.

Antiaging functional molecules include, for example, vitamins, for example vitamin A alcohols, aldehydes, acids and esters. These belong to the class of retinoids which have benefits of antiaging effects on the skin.

Other functional molecules include, for example, the vitamins A, C, E, F, and preferably vitamins and provitamins of the B group. Some of these functional molecules like nicotinamide/niacinamide, panthenol, pantolactone are preferred because they advantageously impart moisturizing and skin calming properties to the microbeads.

Further preferred functional molecules include lipoic acids, and its salts, esters, sugars, nucleosides, nucleotides, peptides and lipids derivatives. These provide antioxidant effects.

Further preferred functional molecules include fatty acids, particularly branched saturated fatty acids and preferably branched eicosanoic acids like methyleicosanoic acid.

The functional molecules are joined to the cellulose nanocrystal and/or the peptide/protein. The bond between the functional molecule and the cellulose and/or the protein can be either covalent or noncovalent bonds based on hydrogen bonding or ionic or van der Waals or hydrophobic interactions, or combinations of noncovalent interactions. Preference is given to noncovalent bonds preferably forming when the functional molecule is spray-dried with the CNC as described in the next section.

### Nanocrystal Coating

The cellulose nanocrystals can be coated before manufacturing the microparticles. As a result, the component(s) of this coating will remain around the nanocrystals, as a coating, in the microparticles. Thus, in embodiments, the nanocrystals in the microparticles are coated.

This is particularly useful to impart a binding effect to the nanocrystals to strengthen the microparticles. Indeed, the very highly porous microparticles may be more brittle, which is generally undesirable and can be counteracted using a binder. In embodiments, this coating is a polyelectrolyte layer, or a stack of polyelectrolyte layers with alternating charges, preferably one polyelectrolyte layer.

Indeed, the surface of the nanocrystals is typically charged. For example, sulfated cellulose I nanocrystals and carboxylated cellulose I nanocrystals and their salts typically have a negatively charged surface. This surface can thus be reacted with one or more polycation (positively charged) that will electrostatically attach itself to, and form a polycation layer on, the surface of the nanocrystals. Conversely, nanocrystals with positively charged surfaces can be coated with a polyanion layer. In both cases, if desired, further polyelectrolyte layers can be similarly formed on top of a previously formed polyelectrolyte layer by reversing the charge of the polyelectrolyte for each layer added.

In embodiments, the polyanions bear groups such as carboxylate and sulfate. Non-limiting examples of such polyanions include copolymers of acrylamide with acrylic acid and copolymers with sulphonate-containing monomers, such as the sodium salt of 2-acrylamido-2-methyl-propane sulphonic acid (AMPS® sold by The Lubrizol® Corporation).

In embodiments, the polycations bear groups such as quaternary ammonium centers. Polycations can be produced in a similar fashion to anionic copolymers by copolymerizing acrylamide with varying proportions of amino derivatives of acrylic acid or methacrylic acid esters. Other examples include quaternized poly-4-vinylpyridine and poly-2-methyl-5-vinylpyridine. Non-limiting examples of polycations include poly(ethyleneimine), poly-L-lysine, poly(amidoamine)s and poly(amino-co-ester)s. Other non-limiting examples of polycations are polyquaterniums. “Polyquaternium” is the International Nomenclature for Cosmetic Ingredients (INCI) designation for several polycationic polymers that are used in the personal care industry. INCI has approved different polymers under the polyquaternium designation. These are distinguished by the numerical value that follows the word “polyquaternium”. Polyquaterniums are identified as polyquaternium-1, -2, -4, -5 to -20, -22, -24, -27 to -37, -39, -42, -44 to -47. A preferred polyquaternium is polyquaternium-6, which corresponds to poly(diallyldimethylammonium chloride).

In embodiments, the coating comprises one or more dyes, which yields a colored microparticles. This dye can be located directly on the nanocrystals surface or on a polyelectrolyte layer.

Non-limiting examples of positively charged dyes include: Red dye #2GL, Light Yellow dye #7GL.

Non-limiting examples of negatively charged dyes include: D&C Red dye #28, FD&C Red dye #40, FD&C Blue dye #1 FD&C Blue dye #2, FD&C Yellow dye #5, FD&C Yellow dye #6, FD&C Green dye #3, D&C Orange dye #4, D&C Violet dye #2, phloxine B (D&C Red dye #28), and Sulfur Black #1. Preferred dyes include phloxine B (D&C Red dye #28), FD&C blue dye #1, and FD&C yellow dye #5.

Substances Interspersed Among the Nanocrystals and/or Deposited on Pore Walls

As explained hereinbelow, the porous microparticles of the invention can be produced using a porogen emulsion and then using spray-drying to aggregate the nanocrystals and the one or more protein together around the porogen droplets and then removing the porogen. It is well-known (and explained below) that emulsions are typically stabilized using emulsifiers, surfactants, co-surfactants and the like, and that such compounds typically arrange themselves within or at the surface of the porogen droplets. These compounds may or may not be removed during the manufacture of the microparticles. If these compounds are not removed, they will remain in the microparticles along the walls of the pores created by porogen removal. Thus, in embodiments, there are one or more substances deposited on the pore walls in the microparticles. In embodiments, these substances are emulsifiers, surfactants, co-surfactants. In embodiments, the one or more protein is one of these substances. In preferred embodiments, gelatin is deposited on the pore walls in the microparticles. Other substances include alginate, albumin, gliadin, pullulan, and dextran.

Similarly, both the continuous phase of the porogen emulsion and the liquid phase of nanocrystal suspension can comprise various substances that will may not be removed during the manufacture of the microparticles. If these compounds are not removed, they will remain in the microparticles interspersed among the nanocrystals. This is useful to impart a binding effect to the nanocrystals to strengthen the microparticles.

### Method of Manufacturing the Microparticles of the Invention

In another aspect of the invention, there is provided a method for producing the above cellulose microparticles. This method comprises the steps of:
a) providing a suspension of cellulose nanocrystals and a solution of the one or more peptide, one or more protein, or mixture thereof;
b) mixing the suspension with the solution to produce a mixture; and
c) spray-drying the mixture to produce the microparticles.

Herein, a “suspension” is a mixture that contain solid particles, in the present case the cellulose nanocrystals, dispersed in a continuous liquid phase. Typically, such suspensions can be provided by vigorously mixing the nanocrystals with the liquid constituting the liquid phase. Sonication can be used for this mixing as can application of a high-pressure homogenizer or a high speed, high shear rotary mixer. A preferred liquid phase is water, preferably distilled water.

The suspension can contain the cellulose nanocrystals in a concentration, for example, from about 0.1 to about 10 wt %, based on the total weight of the suspension. If the viscosity of suspension is high, the suspension can be diluted to ensure good dispersion.

The solution (before mixing with the suspension) can contain the one or more peptide, the one or more protein, or the mixture thereof in a concentration, for example, from about 0.01 wt % to about 50 wt % based on the total weight of the solution. It will be understood that if more than one peptide or protein are present, they may be provided in separate solutions.

The suspension and the solution are mixed together in step b) in a ratio corresponding to the ratio of protein to cellulose nanocrystals desired in the microparticles to be produced.

Mixing the CNC and fibroin solutions together is to be done with minimal shear forces until the solution is homogeneous.

When using fibroin to produce hydrophobic microparticles, the mixture should be spray dried immediately after mixing.

During spray-drying, the solvent of the suspension is evaporated along with any other low boiling-point components. The suspension is first converted into an aerosol that is sprayed into a hot drying chamber where the solvent (water in this case) and other low boiling point chemicals are removed through heat. The remaining dry particulates or microparticles are collected using a cyclone or bag house at the outlet of the dryer.

The noncovalent coupling between the peptide or the protein and the CNC can take place in the dissolved or suspension state before phase separation to form the microbead by spray drying. The solvent is preferably water or a nanoemulsion in water. Noncovalent binding of the protein to the CNC takes place during the process of spray drying in which there is a change of phase from the fluid to the solid state.

After assembly of the microparticle, its morphology can be determined by light and scanning electron microscopy methods. The concentration of the peptide/protein and the spatial distribution of the peptide/protein in the microparticle can be measured by x-ray photoelectron spectroscopy coupled with argon ion depth profiling or by the technique of focused ion beam depth and spatial profiling coupled with spatially resolved energy dispersive analysis by x-ray (EDAX).

In embodiments, in particular those using silk fibroin, after step c), the microparticles can be washed for example with an alcohol, such as methanol or ethanol. This tends to increase the hydrophobicity of the microparticles.

### Incorporating Optional Functional Molecules

As noted above, the functional molecule(s) are joined to the cellulose nanocrystal and/or the peptide/protein. The bond between the functional molecule and the cellulose and/or the peptide/protein can be either covalent or noncovalent bonds based on hydrogen bonding or ionic or van der Waals or hydrophobic interactions, or combinations of noncovalent interactions. Preference is given to noncovalent coupling of the functional molecule with the protein and/or the CNC.

The coupling, covalent or noncovalent, between the functional molecule and the peptide or the protein and/or the CNC can take place in the dissolved or suspended state before phase separation to form the microbead by spray drying.

The solvent is preferably water or a nanoemulsion in water.

In order to bind the functional molecule noncovalently to the protein and/or the CNC, the functional molecule, the peptide/protein and the CNC are all dissolved or suspended in the same solvent (i.e. in the mixture of step b)). Alternatively, the functional molecule and the peptide/protein are dissolved in the same solvent (i.e. in the solution of step a)), and then the combination of both is added to a suspension of CNC (in step b)); or a suspension of CNC is added to the combination of peptide/protein and functional molecule. In another alternative, the functional molecule is dissolved or suspended with the CNC (i.e. in the suspension of step a)) and this combination is added to the peptide/protein solution (during step b)); or the peptide/protein solution is added to the combination of the functional molecule and the CNC suspension.

In embodiments, before being added to the solution or suspension of step a) or the mixture of step b), the functional molecule can first be dissolved in a solvent other than water, especially if the functional molecule is hydrophobic. Alternatively, the functional molecule can first be dissolved in a nanoemulsion.

Noncovalent binding of the functional molecule to the protein and/or the CNC takes place during the process of spray drying in which there is a change of phase from the fluid to the solid state.

If the functional molecule is a dye, then the dye concentration can be determined photometrically and the dye distribution at the surface can be determined by hyperspectral imaging.

Since the protein is typically a charged molecule, a functional molecule such as a dye bearing a charge opposite to that of the protein can be assayed by measuring the extinction spectrum of the microbead. In this case, it is possible to determine the charge density of the protein/CNC microbead and the charge efficiency, which is the percentage of functional dye molecules attached to the protein/CNC microbead.

### Manufacturing Porous Microparticles

When porous microparticles are desired, this method can be slightly modified. The resulting method comprises the steps of:
a) providing a suspension of cellulose nanocrystals, a solution of the one or more peptide, one or more protein, or mixture thereof, and an emulsion of a porogen, wherein the solution of the one or more peptide, one or more protein, or mixture thereof either is part of the emulsion or stands alone;
b) mixing the suspension with the solution and the emulsion to produce a mixture comprising a continuous liquid phase in which droplets of the porogen are dispersed, the cellulose nanocrystals are suspended, and the one or more peptide, one or more protein, or mixture thereof is dissolved;
c) spray-drying the mixture to produce microparticles; and
d) if the porogen has not sufficiently evaporated during spray-drying to form pores in the microparticles, evaporating the porogen or leaching the porogen out of the microparticles.

During spray-drying, the nanocrystals arrange themselves around the porogen droplets. Then, the porogen is removed (creating pores within the microparticles. Porogen removal can happen spontaneously during spray-drying (if the porogen is sufficiently volatile) or otherwise, the porogen is removed in subsequent step d).

Herein, an “emulsion” is a mixture of two or more liquids that are immiscible, in which one liquid, called the dispersed phase, is dispersed in the form of droplets in the other liquid, called the continuous phase. All the above types of the emulsions can be used in the present method. However, macroemulsions that can be used in the present method are limited to those macroemulsions in which the droplets of the dispersed phase have a diameter of at most about 5 μm.

Emulsions are typically stabilized using one or more surfactants, and sometimes co-surfactants and co-solvents, that promote dispersion of the dispersed phase droplets. Microemulsions form spontaneously as a result of ultralow surface tension and a favorable energy of structure formation. Spontaneous formation of the microemulsion is due to the synergistic interaction of surfactant, co-surfactant and co-solvent. Microemulsions are thermodynamically stable. Their particle size does not change over time. Microemulsions can become physically unstable if it is diluted, acidified or heated. Nanoemulsions and macroemulsions do not form spontaneously. They must be formed by application of shear to a mixture of oil, water and surfactant. Nanoemulsions and macroemulsions are kinetically stable, but thermodynamically unstable: their particle size will increase over time via coalescence, flocculation and/or Ostwald ripening.

Step b) of providing an emulsion of a porogen includes mixing two liquids that are immiscible with each other, optionally together with emulsifiers, surfactant(s), and/or co-surfactant(s) as needed to form an emulsion in which droplets of one of the two immiscible liquids will be dispersed in a continuous phase of the other of the two immiscible liquids.

Herein, the term “porogen” refers to those components of the dispersed phase (one of the immiscible liquids, the emulsifiers, surfactant(s), and/or co-surfactant(s), as well as any other optional additives) that are present in the droplets at steps a) and b) and that are removed from the microparticles at steps c) and/or d) thus forming pores in the microparticles. Typically, the porogen includes the liquid (among the two immiscible liquids contained in the emulsion) that forms the droplets. The porogen may also include emulsifiers, surfactant(s), and/or co-surfactant(s); although some of those may also be left behind (i.e. not be a porogen) as explained above.

In step c), the spray-drying causes the cellulose nanocrystals to assemble around and trap the porogen droplets and to aggregate into microparticles. Furthermore, if the porogen has a sufficiently low boiling point, spray-drying will then cause the evaporation of the porogen droplets creating pores in the microparticles. If the porogen does not have a sufficiently low boiling point, it will only partially evaporate or not evaporate at all during spray-drying step c). In such cases, to form the desired pores, the porogen will be removed from the microparticles during step d). Hence, step d) is optional. It need only be carried out when the porogen has not (or not sufficiently) evaporated during spray-drying.

Examples of porogens that typically evaporate during spray-drying, i.e. “self-extracting porogens”, include:
terpenes, such as limonene and pinene, camphene, 3-carene, linalool, caryophyllene, nerolidol, and phytol;
alkanes, such as heptane, octane, nonane, decane, and dodecane;
aromatic hydrocarbons, such as toluene, ethylbenzene, and xylene;
fluorinated hydrocarbons, such as perfluorodecalin, perfluorhexane, perfluorooctylbromide, and perfluorobutylamine.

Step d) is the evaporation of the porogen or leaching of the porogen out of the microparticles. This can be achieved by any method as long as the integrity of the microparticles is maintained. For example, evaporation can be achieved by heating, vacuum drying, fluid bed drying, lyophilization, or any combination of these techniques. Leaching can be achieved by exposing the microparticles to a liquid that will dissolve the porogen (i.e. it is a porogen solvent) while being a non-solvent for the cellulose I nanocrystals.

### Using Fibroin as a Protein in the Microparticles

The fibroin to be used in the microparticles can be any fibroin. Non-limiting examples include fibroin obtained from gummy (still containing sericin) silk cocoons and sheets, as well as degummed silk tops, hankies, and bricks as well as cosmetic grade silk powders.

Obtaining fibroin from gummy cocoons and sheets required two process steps: degumming followed by fibroin dissolution. In contrast, obtaining fibroin from degummed silk tops, hankies, and bricks and cosmetic grade silk powders required only one process step: fibroin dissolution. Methods for degumming and fibroin dissolution are well-known to the skilled person.

As noted above, fibroin allows producing hydrophobic microparticles even when used in a concentration as low as 0.5 wt %, based on the weight on the microparticle. However, as also noted above, the mixture obtained at step b) of the above method should be spray dried as soon as possible. Indeed, leaving the suspension to stand for more than 3 days will not result in hydrophobic microparticles.

### Advantages of the Microparticles of the Invention

In embodiments, the microparticles of the invention can have one or more of the following advantages.

They combine the benefits of peptides and/or proteins whilst hosting them in a biodegradable matrix that retains the structural integrity of the microparticles. The Applicants have discovered that this can be advantageously accomplished by blending peptides and/or proteins with cellulose nanocrystals (CNCs) by the process of spray drying to make CNC-protein microbeads. This manufacturing method advantageously requires few steps.

As noted above, in some embodiments (including microparticles silk fibroin), it is possible to tailor the properties of the microparticles from hydrophilic to hydrophobic/lipophobic. This is advantageous as there is a need in the cosmetic industry for microbeads that exhibit these latter properties. Indeed, such microparticles are beneficially compatible with hydrophilic or lipophilic host media like oils, waxes and many petroleum-based polymers. More details are cosmetics preparations comprising the microparticles of the invention are provided in the next section.

In particular, the Applicant has surprisingly discovered that the combination of SF with carboxylated or sulfated CNC, when spray dried together, yields composite carboxylated cellulose/SF or sulfated cellulose/SF microbeads that are hydrophobic and lipophilic. The discovery is surprising because the literature on cellulose/SF composites, including cellulose nanofibers and cellulose nanocrystals, indicates that SF in combination with cellulosics, are hydrophilic and in some cases show enhanced moisture retention. The discovery is even more important because incorporation of SF as described below reduces the number and complexity of coating steps required to convert a hydrophilic microbead into a lipophilic microbead.

In the microparticles of the invention, the bonds formed between CNCs and the peptides are noncovalent, i.e. there are preferably no covalent bonds. The formation of covalent chemical bonds between a protein and CNC is undesirable for several reasons. For example, the Maillard reaction confers an undesirable deep brown coloration to the protein-CNC composite. This makes such composites unpleasing for applications in cosmetics.

It is advantageous that the microparticles are naturally and sustainably sourced. Indeed, the cosmetic and personal care industry is moving towards the creation of products that are “naturally sourced”. This term is difficult to define, and the ISO group has approached the problem by defining a “natural index”. The natural index is a value indicating the extent to which a cosmetic ingredient meets the definition of natural ingredients from ISO 16128-1:2016, clause 2. The value can be construed as varying between 0 and 1, where 1 can be interpreted as 100% natural (of “organic” origin). The cosmetics industry is pressuring suppliers of ingredients to use sustainable manufacturing methods in the production of ingredients, to ensure a high natural index and to exclude GMO additives. Accordingly, there is a need for lipophilic/hydrophobic microbeads that are derived in whole or in part from natural sources, which the present invention provides.

The microparticles of the invention can bring new benefits to consumers by virtue of desirable changes for texturizing, ease of formulation for enhanced skin feel, for desirable optical properties like soft focus, and for dermocosmetics.

In embodiments, the microparticles of the invention can also bring additional benefits to the skin by means of the functional molecules that can be carried. As noted above, these benefits include for example protection against ultraviolet light and blue light, antioxidant and anti-aging properties, moisturizing effects, and color.

### Uses of the Microparticles of the Invention

The microparticles of the invention can be used in a cosmetic preparation. For example, they can replace plastic microbeads currently used in such preparations. Thus, in another aspect of the invention, there is provided a cosmetic preparation comprising the above microparticles and one or more cosmetically acceptable ingredients.

The nature of these cosmetically acceptable ingredients in the cosmetic preparation is not crucial. Ingredients and formulation well-known to the skilled person may be used to produce the cosmetic preparation.

Herein, a “cosmetic preparation” is a product intended to be rubbed, poured, sprinkled, or sprayed on, introduced into, or otherwise applied to the human body for cleansing, beautifying, promoting attractiveness, or altering appearance. Cosmetics include, but are not limited to, products that can be applied to:
the face, such as skin-care creams and lotions, cleansers, toners, masks, exfoliants, moisturizers, primers, lipsticks, lip glosses, lip liners, lip plumpers, lip balms, lip stains, lip conditioners, lip primers, lip boosters, lip butters, towelettes, concealers, foundations, face powders, blushes, contour powders or creams, highlight powders or creams, bronzers, mascaras, eye shadows, eye liners, eyebrow pencils, creams, waxes, gels, or powders, setting sprays;
the body, such as perfumes and colognes, skin cleansers, moisturizers, deodorants, lotions, powders, baby products, bath oils, bubble baths, bath salts, body lotions, and body butters;
the hands/nails, such as fingernail and toe nail polish, and hand sanitizer; and
the hair, such as shampoo and conditioner, permanent chemicals, hair colors, hairstyling products (e.g. hair sprays and gels).

A cosmetic may be a decorative product (i.e. makeup), a personal care product, or both simultaneously. Indeed, cosmetics are informally divided into:
“makeup” products, which are primarily to products containing color pigments that are intended to alter the user's appearance, and
“personal care” products encompass the remaining products, which are primarily products that support skin/body/hair/hand/nails integrity, enhance their appearance or attractiveness, and/or relieve some conditions that affect these body parts.
   Both types of cosmetics are encompassed within the present invention.

A subset of cosmetics includes cosmetics (mostly personal care products) that are also considered “drugs” because they are intended for use in the diagnosis, cure, mitigation, treatment, or prevention of disease or intended to affect the structure or any function of the body of man or other animals. Examples include antidandruff shampoo, deodorants that are also antiperspirants, products such as moisturizers and makeup marketed with sun-protection claims or anti-acne claims. This subset of cosmetics is also encompassed within the present invention.

Skin feel is an extremely important property of cosmetic preparation. Preparations with good, or preferably excellent, skin feel are preferred by customer.

A microparticle that absorbs sebum is desirable because it makes the skin look less shiny and therefore more natural (if the microparticle is non-whitening)—this is referred to as the mattifying effect.

There is a need in the cosmetic industry for microbeads that are hydrophobic and simultaneously lipophilic (as per the definition given above). A lipophilic chemical compound will have a tendency to dissolve in, or be compatible with fats, oils, lipids, and non-polar organic solvents like hexane or toluene. Such microbeads have the advantage that they are more easily formulated in water-in-oil emulsions, and in other largely lipophilic media (like lipsticks).

Due to environmental concerns, plastic microbeads, including porous plastic microbeads, are banned or are being banned throughout the world, thus there is a need to replace them with microparticles that offer the same benefits (tunable oil uptake and mattifying effect), but are friendlier to the environment. Microparticles with improved oil uptake, with lipophilicity, and with improved skin feel, such as those provided here, are thus advantageous to the cosmetics industry. They can replace plastic microbeads whilst retaining their benefits.

### Definitions

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed.

No language in the specification should be construed as indicating any non-claimed element as essential to the practice of the invention.

Herein, the term “about” has its ordinary meaning. In embodiments, it may mean plus or minus 10% or plus or minus 5% of the numerical value qualified.

Unless otherwise defined, all technical and scientific terms used herein have the same meaning as commonly understood by one of ordinary skill in the art to which this invention belongs.

Other objects, advantages and features of the present invention will become more apparent upon reading of the following non-restrictive description of specific embodiments thereof, given by way of example only with reference to the accompanying drawings.

### Description of Illustrative Embodiments

The present invention is illustrated in further details by the following non-limiting examples.

### Preparation of Various Cellulose Nanocrystals (CNC) Suspensions

### CNC Suspension #1—Carboxylated CNC

The cellulose nanocrystal suspension used as a starting material below was produced using the method provided in International patent publication no. WO 2016\015148 A1.

Briefly, dissolving pulp (Temalfa 93) was dissolved in 30% aqueous hydrogen peroxide and heated to reflux with vigorous stirring over a period of 8 hours. The resulting suspension was diluted with water, purified by diafiltration and then neutralized with aqueous sodium hydroxide.

The resulting concentrated stock suspension of sodium carboxylate nanocrystalline cellulose (cCNC) typically consisted of 4% CNC in distilled water. This suspension was used as is or diluted with distilled water as needed for use in the Examples below.

sCNC Suspension #2—Sulfated CNC

Sulfated CNC was prepared according to the method of Revol et al. (Dong, X.; Revol, J.-F.; Gray, D., Effect of microcrystallite preparation conditions on the formation of colloid crystals of cellulose. Cellulose 1998, 5 (1), 19-32)

### Examples 1 to 7—Fibroin/Cellulose Microparticles

### Preparation of Fibroin Solution #1

1-2 g of silk fibroin (from Ikeda Corporation—cosmetic grade fibroin powder) was added to 5.55 g CaCl₂, 4.6 g ethanol, 7.2 g distilled water (molar ratio of CaCl₂):Ethanol:H₂O is 1:2:8) at 80° C. (Caution: the Ajisawa solvent mixture generates a lot of heat). Silk fibroin was pressed down so it was fully immersed in the solvent. After 20-30 min, the fibroin seemed completely dissolved and the solution became transparent with a tint of yellow color.

The fibroin solution was pipetted to cellulose dialysis tube and dialysed against distilled water in a 3.5 L glass beaker. The water was changed every hour for the first day and then changed every half a day. The whole dialysis process took three days.

The fibroin concentration of the solution in the dialysis tube after dialysis was 1.5-2.0 wt %.

Using Fibroin Obtained from Other Sources or Dissolved Using Other Reagents

The present inventors have used fibroin obtained from gummy (still containing sericin) silk cocoons and sheets, as well as degummed silk tops, hankies, and bricks as well as cosmetic grade silk powders. Silk from India, Laos, Japan, and China can be used. These starting materials were used to produce fibroin solutions by the various methods described below. In all cases, the obtained fibroin produced hydrophobic fibroin-containing cellulose microparticles with hydrophobicities similar to those reported herein when using fibroin solution #1.

To obtain fibroin from gummy cocoons and sheets required two process steps: degumming followed by fibroin dissolution. In contrast, to obtain fibroin from degummed silk tops, hankies, and bricks and cosmetic grade silk powders required only one process step: fibroin dissolution.

### Degumming—Alkaline Method Using Sodium Carbonate

An aqueous solution containing sodium carbonate at a concentration of 2.12 g sodium carbonate/L in water was boiled. Once the water was boiling and homogeneous, the silk was added, and the solution was boiled for 15-30 min, stirring occasionally to ensure even removal of sericin. Then, the fibers were removed from the boiling liquid and rinsed in cold deionized water. Excess water was wrung out and the fiber was added to 1 L of deionized water, stirring occasionally for 20 min. The fibers were removed from the water and excess water was squeezed out. The water was discarded and this rinsing process was repeated two more times to thoroughly wash out the sodium carbonate. When the fibroin was removed from the water for the last time, any excess water was squeezed out and the fibers were spread out onto a clean piece of aluminum foil and allowed to dry overnight. These fibers were then stored at room temperature until used.

### Fibroin Dissolution

Several methods have been used to dissolve fibroin:
the LiBr method (9.3M LiBr aqueous solution),
the Ajisawa method (CaCl₂/EtOH/H₂O), and
   all of which are exemplified below. Typically, silk powder dissolved more readily than silk fiber and lower temperatures/less time was needed for the fibroin to go into solution.

To test if the fibroin was fully dissolved, a visual inspection was done before moving on to any purification steps (which are also described below). The fibroin was considered totally dissolved when there was no visible sign of suspended particles.

After purification, fibroin solutions were stored in refrigerator for up to 10 days.

### The LiBr Method

A 9.3M solution of LiBr was prepared, making sure to add LiBr to the water slowly as this is an exothermic process. The required amount of degummed fibroin was packed into the smallest container that could fit all the components. The LiBr solution was added on top of the silk (the LiBr solution must be introduced in the container after the silk!) at a concentration of 4 mL of 9.3M LiBr solution per gram of degummed fibroin. The mixture was allowed to stand in a 55-60° C. oven for 4 h until it became highly viscous, but no longer contained any visible fibers. The resulting solution was placed into dialysis tubing and dialyzed against water (1 L of water/12 mL of fibroin solution). The water was replaced after 1 h, 4 h, that evening, the next morning and the next night, and the morning of the following day (i.e. six changes of water within 48H) to obtain the desired fibroin solution.

Sometimes, when the fibroin was obtained from cocoons, solid detritus was present in the fibroin solution. In such cases, the detritus was removed using a centrifuge at 9000 rpm for 20 min, preferably at 4° C. (although room temperature also worked).

### The Ajisawa Method

A solution of CaCl₂/EtOH/H₂O at molar ratios of 1:2:8 was prepared. Between 8-9 g of solution per gram of silk fibroin were used. The silk was fully wetted by the solution and then placed the into an oven at a temperature between 50-100° C. until all the fiber was dissolved (typically, it took 20 to 120 min).

The solution containing dissolved fibroin was purified using one of two methods: either dialysis or a size exclusion column (sephadex G-25 desalting resin, from GE Healthcare). If using a size exclusion column, the solution was diluted with water (10 g of water/1 g of fibroin) and then the solution was run through the desalting column. If using dialysis, the solution was transferred to dialysis tubing and dialyzed against water (using around 1 L of water/1 g of fibroin). The water was replaced every hour for the first day and then every half a day over 48 h.

### Measuring the Hydrophobic Response of the Fibroin/Cellulose Microparticles

A simple qualitative determination of a hydrophobic response is the measure of its tendency to repel water. Accordingly, hydrophobic response of the microparticles can be determined visually in either of two ways:
By placing a sample of the powder onto a glass microscope slide and adding water to see if water wets the powder or is repelled by the powder.
By placing a sample of the powder (˜10 mg) into a 0.5 dram vial to which 1 mL of water is then added. The capped vial is then shaken for 5 sec. As the mixture settles the powder will either float on the surface of the water (hydrophobic measure) or will disperse in the water (hydrophilic measure).

The qualitative measure of hydrophobicity also is then tested in a water-in-oil emulsion designed for this purpose. The emulsion composition and procedure are as follows:

| Phase | INCI Name | Trade Name | wt % |
|---|---|---|---|
| 1 | C12-15 Alkyl Benzoate | Jeechem TN | 20 |
| 1 | Lauryl PEG-9 Polydimethylsiloxyethyl | KF-6038 | 4 |
| | Dimethicone | | |
| 1 | Acrylates/Ethylhexyl Acrylate/Dimethicone | KP-578 | 4 |
| | Methacrylate Copolymer | | |
| 2 | Cellulose (and) Fibroin | ChromaPur | 2 |
| | | Soie ™ | |
| 3 | Water | Water | 62 |
| 3 | 1,3-Butylene Glycol | 1,3-Butylene | 3 |
| | | Glycol | |
| 3 | NaCl | NaCl | 1 |
| 3 | EtOH | 95% EtOH | 4 |

| | |
|---|---|
| TOTAL | 100% |

The emulsion was prepared as follows:
1. The ingredients of phase 1 were mixed on Rayneri mixer equipped with saw tooth blade at 400 rpm for 5 mins at 75° C.
2. The ingredient of phase 2 was added to phase 1 and mixed for 2×5 min at 500 rpm.
3. The ingredients of phase 3 were combined and mixed on magnetic stir plate at 400 rpm while heating to 75° C.
4. Phase 3 was slowly added to phase 1+2 while increasing agitation speed from 600 rpm to 1200 rpm.
5. Once the emulsion was formed, the speed was increased to 2500 rpm for 5 mins while heating at 75° C.
6. The emulsion was allowed to cool to room temperature while slowing mixing at 300 rpm

Cellulose microparticles with high hydrophilicity agglomerate in water-in-oil emulsions due to their preference for the water phase, which exists as discrete droplets in these emulsion systems. Indeed, when the individual microparticles move into the water phase, they aggregate in the water droplets resulting in agglomerates. Hydrophilic microparticles therefore end up agglomerating into progressively larger particles until they are easily seen visually. In contrast, aggregation in water-in-oil emulsions is not observed for hydrophobic microparticles.

### Example 1—Hydrophobic Fibroin/Cellulose Microparticles from Carboxylated CNC with Silk Fibroin

CNC suspension #1 (2.17 wt % CNC) was mixed in with fibroin solution #1 (1.8 wt %) such that the final fibroin content to CNC content was 2 wt %. Mixing the CNC and fibroin solutions together was done with minimal shear forces ensuring efficient stirring for the volume size. Mixing was performed until the solution was homogeneous within 10 min. The suspension was immediately spray dried (Techni Process spray dryer model SD-1; inlet temperature 190° C., outlet temperature 89-92° C., nozzle pressure 2 bar, differential pressure 180 mm WC). After spray drying, the resulting free-flowing white powder may be washed with an alcohol like ethanol, followed by 30 min in 80° C. oven to increase the hydrophobic effect. The obtained microparticles had a 2 wt % silk fibroin content.

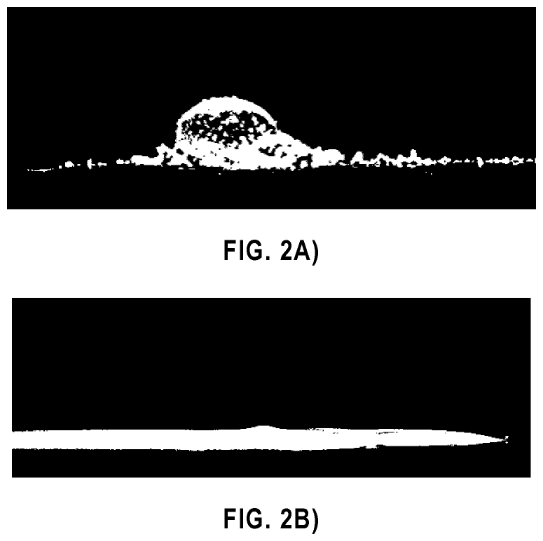

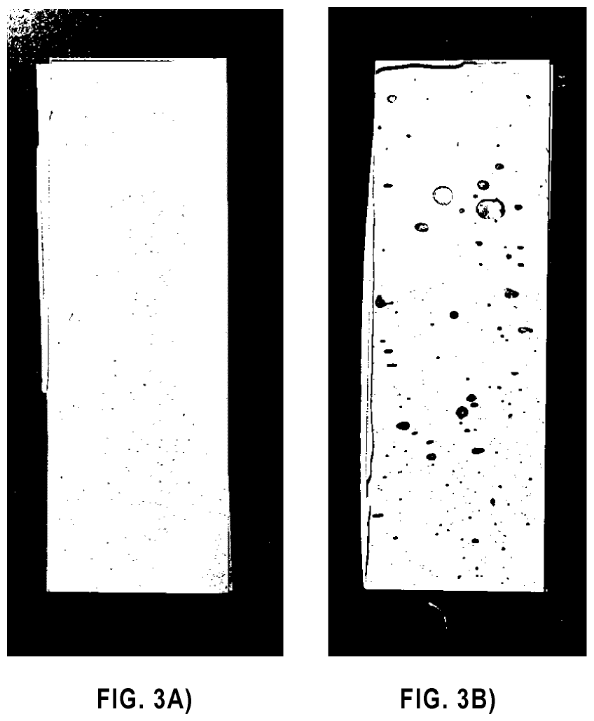

FIG. 2A) shows a sample of the powder obtained to which water was added. It can clearly be seen that the powder sat on the surface of the water droplet, rather than being wetted. This indicates that the microparticles are hydrophobic.

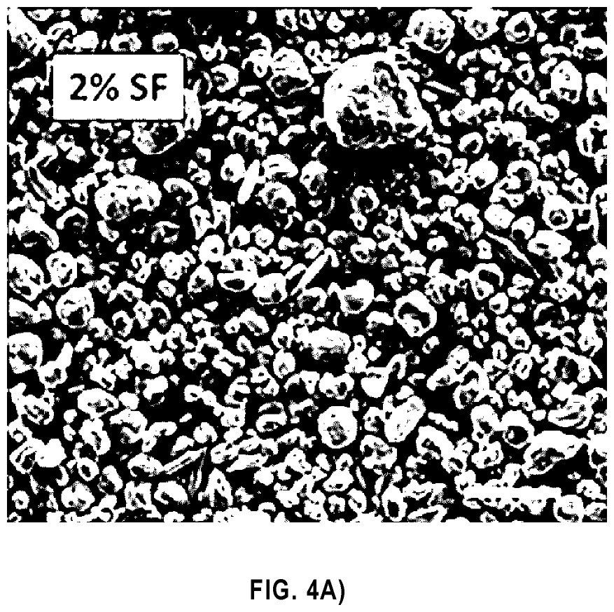

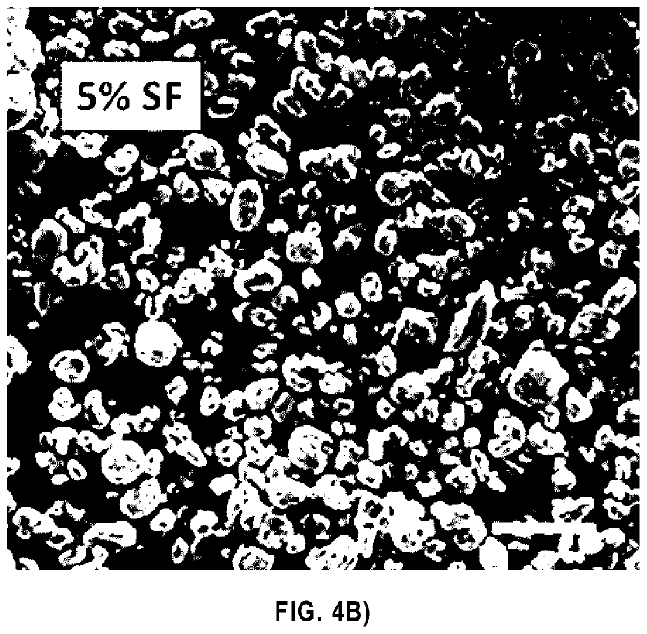

FIG. 3A) shows the powder obtained mixed the above water-in-oil emulsion. No aggregates can be observed, indicating that the microparticles are hydrophobic.

### Example 2—Hydrophobic Fibroin/Cellulose Microparticles with Silk Fibroin Content to Carboxylated CNC Content Ranging From 0.5 wt % to 50 wt %

Hydrophobic microparticles were obtained in the same manner as described in Example 1.

More specifically, silk fibroin solution (2 wt-%) was added to CNC suspension #1 (0.5 wt-%) under vigorous stirring to obtain solutions of 5 wt-%, 10 wt-%, 20 wt-% and 50 wt-% SF respectively. The resulting suspensions were spray dried on a Buchi Mini Spray Dryer model B-191 (Inlet temperature 175° C. and outlet temperatures of 100° C., 30% pump speed, 70% aspirator). Some samples were treated with methanol to increase the proportion of beta-pleated sheet SF in the microbead.

The obtained powders were hydrophobic.

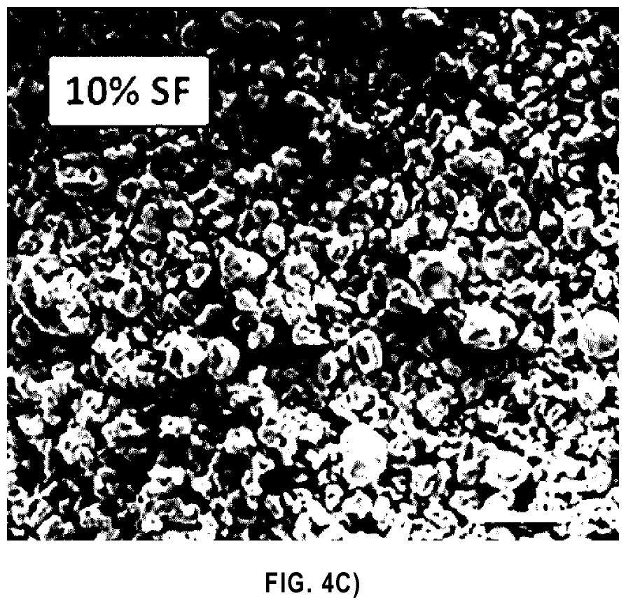

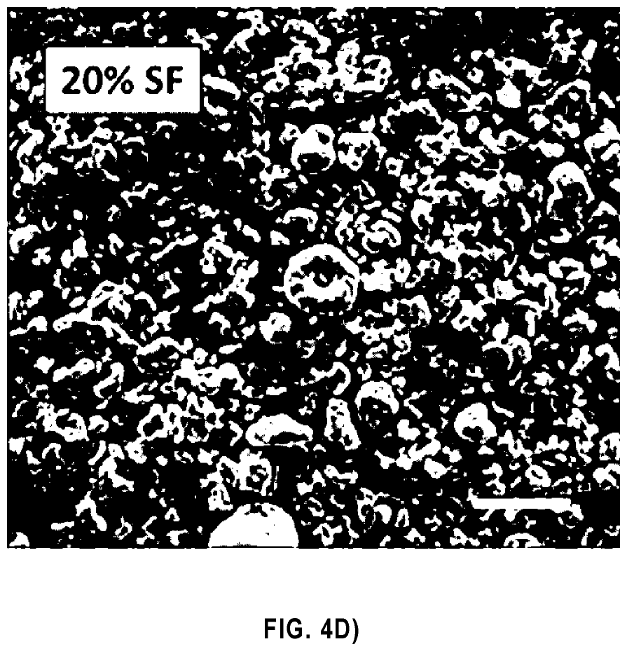

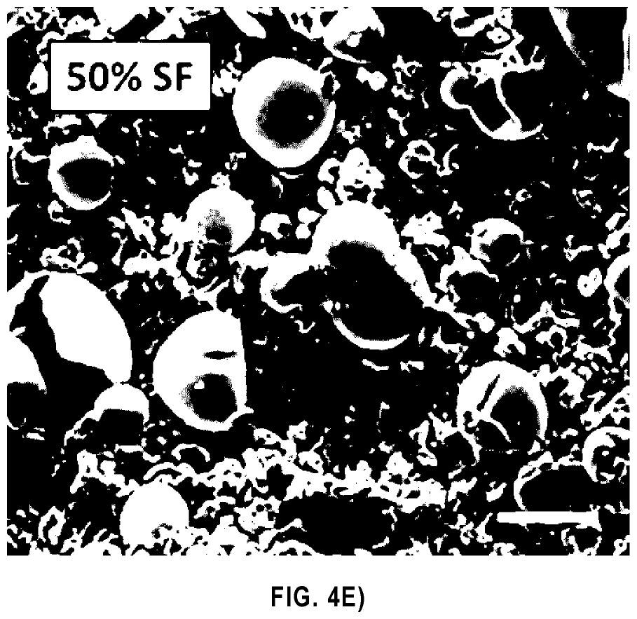

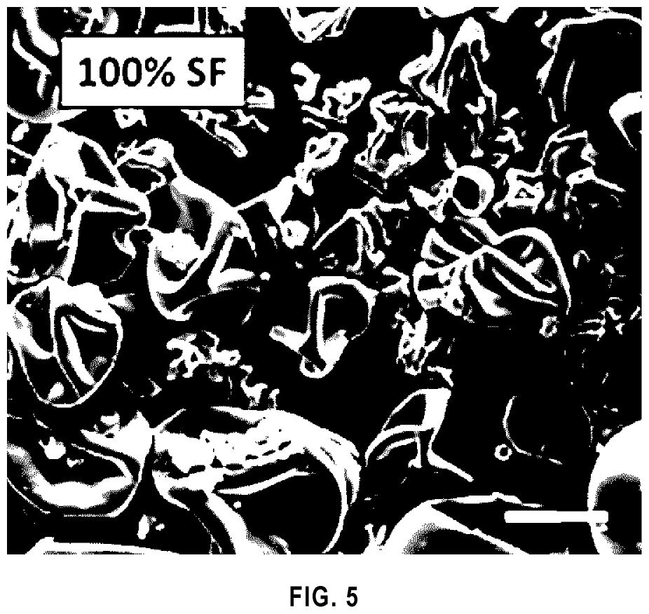

FIG. 4 shows SEM images of the microparticles obtained.

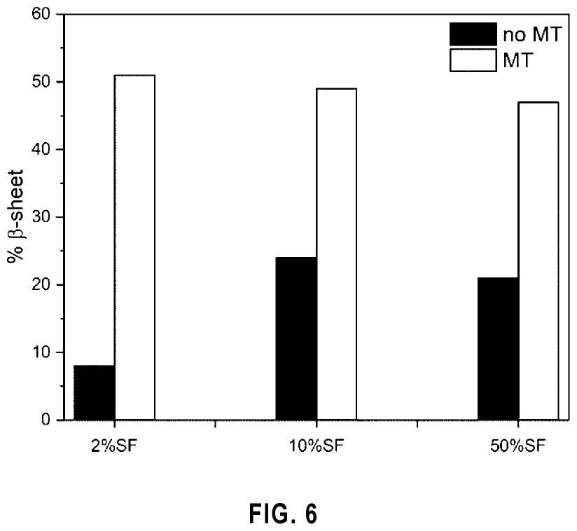

For comparison, FIG. 5 shows microparticles obtained by spray-drying silk fibroin only (i.e. without CNC).

The presence of SF in the beta-pleated sheet form appears to be linked to the hydrophobic effect that is conferred when SF is mixed with CNC to make the hybrid SF/CNC microparticles. It would appear that a portion of the SF must be concentrated at the surface or near sub-surface of the hybrid microbead, else the microbead may be wetted by water.

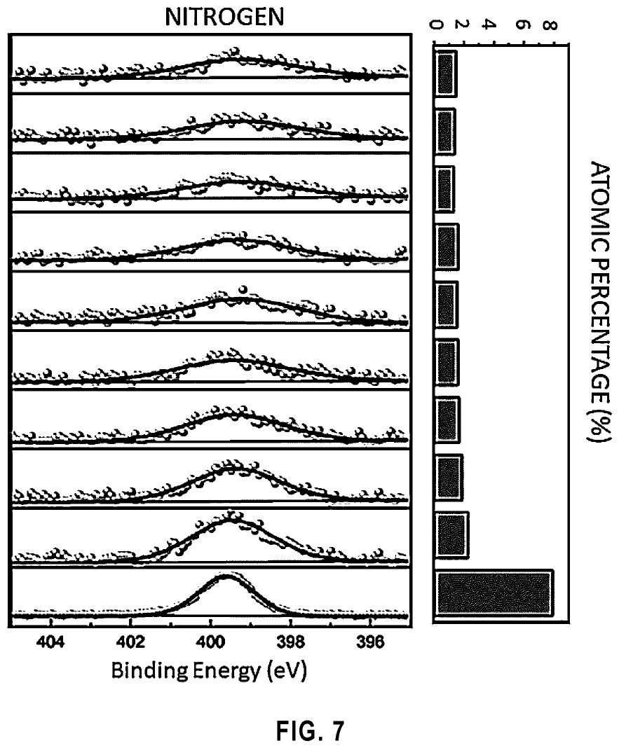

The relative amount of beta-pleated sheet SF in the microbeads was determined by analyzing the percentage of SF chain conformations that contribute infrared absorption in the region 1580 to 1720 cm⁻¹ in the amide stretching region. FTIR spectra were measured with a Bruker ALPHA FTIR spectrometer (Bruker Optics Inc., Billerica, USA) for microsphere powders in the spectral region of 400 cm⁻¹ to 4000 cm⁻¹, acquired with 60 scans at a nominal resolution of 4 cm⁻¹. The relative contributions of beta-pleated sheet, beta-turns, alpha helix, random coil and aggregated strands were determined by standard curve-fitting with Gaussian deconvolution (OriginPro 2018b software (OriginLab, Northampton, USA). Methanol was used to induce the conformational transition of silk fibroin to the insoluble beta-sheet state. FIG. 6 shows the percentage of beta-pleated sheet in samples of microbeads before treatment with methanol (no MT) and after treatment with methanol (MT). The figure shows that methanol treatment increases the percentage of beta-pleated sheet SF in the microbead.

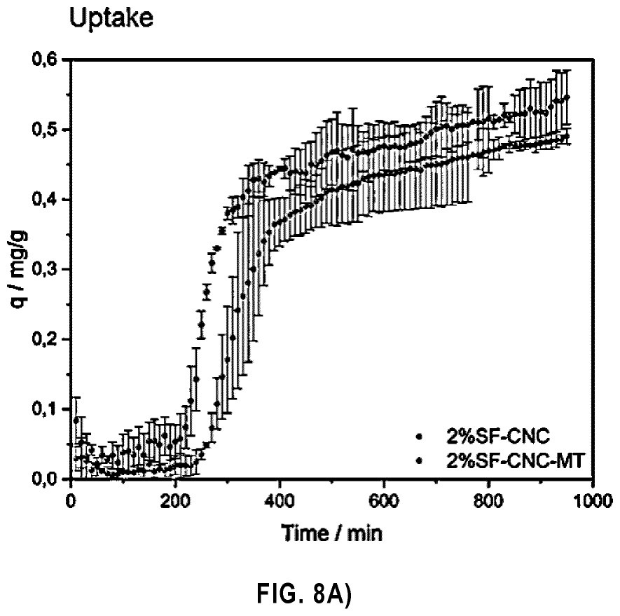

X-ray Photoelectron Spectroscopy (XPS) is a highly sensitive surface analysis method that probes the top 10 nm of a surface. When combined with sputtering or etching sources to remove material slowly between analysis cycles without damaging underlying material, depth-profiling XPS enables high-resolution chemical analysis. The spatial location of SF in a 2% SF/CNC microbead sample can be determined by depth-profiling XPS. XPS measurements were performed with a Thermo Scientific K-Alpha spectrometer. An argon ion gun with an energy of 500 eV and 1.00 μA current was used for depth profiling, which was performed for 300 s with 10 cycles. XPS was performed on each etching level with the flood gun on. The X-ray emission angle was 90 degrees with respect to the specimen surface. The diameter of the analyzed area was 400 μm. It was estimated that 10 min of etching corresponded to an etching depth of 1 μm. Spectra were deconvolved, and the resulting curves attributed to the different kinds of bonds according to their binding energy. Integration of the resolved curves allowed calculation of the atomic nitrogen percentage. FIG. 7 shows the depth profile of the nitrogen 1 s peak associated with SF at 2% loading in the microbead. Depth profiling was achieved by measuring the binding energy intensity peaked at 399.7 eV for nitrogen as a function of time. Spectra were referenced to the C1s peak of aliphatic carbon at a binding energy of 285.0 eV. In the figure, position 1 refers to the surface of the microbead without Ar+ erosion. Positions 2 through 10 are 5-minute increments of Ar+ erosion, and therefore are measures of the protein content in the interior of the microbead. The figure shows that SF in 2% SF/CNC microbeads is more concentrated at the surface and then is more uniformly distributed in the interior of the microbeads in the sample.

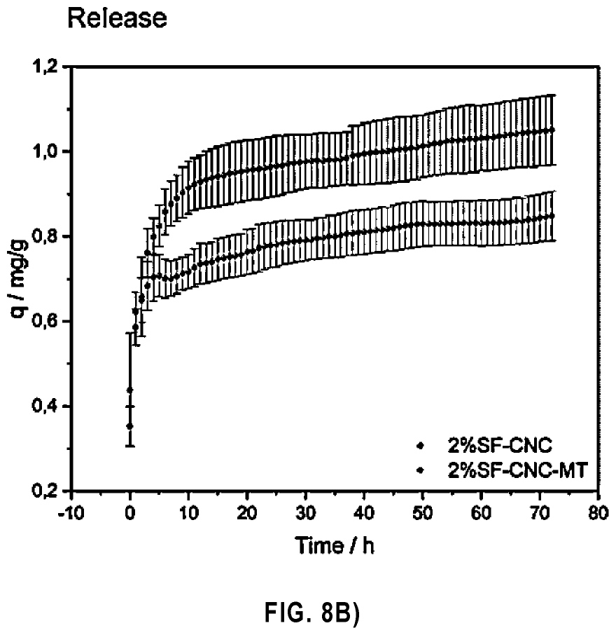

The water-soluble dye molecule, methylene blue (MB), is taken up almost instantaneously by CNC microbeads that contain no SF. Therefore, another measure of the hydrophobic barrier properties of SF/CNC microbeads is to measure MB take-up. Methylene blue uptake and release on SF/CNC microbeads was measured on a Thermo Scientific™ Evolution™ 260 Bio UV-Vis spectrophotometer (Fisher Scientific Company, Ottawa, Canada). Methylene blue was obtained from Alfa Aesar (Heysham, UK), methanol was obtained from Fisher Chemicals (Fair Lawn, USA) and acetone from Anachemica (Mississauga, Canada). Uptake and release studies were performed in the same way on non-methanol treated and on methanol treated microspheres. For methanol treatment, SF/CNC microspheres (100 mg) were left overnight in an aqueous methanol solution (80 wt-%, 100 mL), filtered and washed with acetone. For uptake monitoring, SF/CNC microspheres (5 mg) were immersed in methylene blue solution (10 mg/L, 3 mL) and mixed. The measurement was performed for 16 h, measuring every 10 min at a wavelength of 665 nm and a reference wavelength of 750 nm. For release monitoring, samples were prepared by immersing SF/CNC microbeads (100 mg) in methylene blue solution (78 mg/L, 45 mL) overnight, filtering and washing with acetone. The measurement was done by immersing the dyed microbeads (5 mg) in water (3 mL), mixing and measuring every hour for 72 h at a wavelength of 665 nm and a reference wavelength of 750 nm. FIG. 8 shows the take-up and release of MB by 2% SF/CNC microbeads with and without treatment by methanol. Compared with CNC microbeads with no SF, MB begins to be taken up by the SF hybrid beads only after some 200 hours (no methanol treatment, no-MT) and after about 250 hours (MT). The release of MB occurs largely from the surface of the microbead. This is evident in the almost instantaneous release kinetics (right-hand side curves) and rapid plateau. More dye is released from the no-MT beads than from the MT beads, consistent with the lower quantity of beta-pleated sheet SF in the no-MT sample.

### Example 3—Hydrophobic Fibroin/Cellulose Microparticles from Sulfated CNC with 2% Silk Fibroin

A hydrophobic cellulose microbead was prepared using silk fibroin and sulfated NCC.

Accordingly, 70 mL of a 0.68 wt % solution of sulfated CNC suspension #2 (0.476 g sNCC) was stirred at 200 rpm on a stir plate with a magnetic stirring bar. Then 0.464 mL of a 2.05 wt % (9.52 mg) of fibroin solution #1 was slowly added under constant stirring. Stirring continued for 10-15 min. until the fluids were homogeneous. The suspension was then spray dried (Buchi spray dryer model B191: inlet temperature 165-185° C., pump speed 30%, aspirator 70%, air pressure 600N1/h). A free-flowing white powder was produced.

When tested as described above, the powder was found to be hydrophobic.

### Example 4—Porous Hydrophobic Fibroin/Cellulose Microparticles

This Example shows that porous hydrophobic fibroin/cellulose microparticles can be produced when the nanoemulsion is prepared from a non-volatile oil/surfactant system.

A 400 nm nanoemulsion was prepared as follows: 0.021 g Montanov™ 82 (SEPPIC) was dissolved in 470 ml distilled water at 60° C. 10 g alkyl benzoate was then poured into Montanov™ 82 solution and stirred at 60 C for 10 min at 1000 rpm. The mixture was then sonicated at 60% amplitude (Sonics® Vibra-Cell) in iced water bath for 20 min to produce nanoemulsion with an average droplet diameter of 400 nm.

300 mL of CNC suspension #1 (1.90 wt %) was poured into the above emulsion and mixed at 300 rpm for 10 min. 28 ml of fibroin solution #1 (1.88 wt %) was poured into the above mixture and stirred at 300 rpm for 10 min before spray-drying. The spray drier parameters were set as follows: inlet temperature 185 C, outlet temperature: 85 C, nozzle pressure 1.50 bar, differential pressure 180 mmWc, and nozzle air cap 70. The process yielded a dried free-flowing white powder.

To remove the embedded porogen (i.e. alkyl benzoate) and induce fibroin 3-sheet formation, a 2 g lot of the spray dried microbeads was added to 40 mL ethanol and mixed for 3 min before being centrifuged at 1200 rpm for 6 min. This step was repeated one time, discarding the supernatant liquid each time. The sample was then dispersed into 20 mL ethanol. The dispersion was poured into a 500 mL evaporating flask and dried in a vacuum of 25 mbar (Heidolph rotary evaporator; (Basis Hei-Vap ML)) at 60° C. with rotation at 70 rpm. A white free-flowing powder was formed after 1 hour.

The powder did not mix well with water and stayed on the water surface when added to water, indicating that the microparticles were hydrophobic.

Oil uptake was measured using the fluid saturation method as described in US standard ASTM D281-84. The oil uptake was measured to be 195 ml/100 g.

### Example 5—Porous Hydrophobic Fibroin/Cellulose Microparticles

This example shows that porous hydrophobic silk fibroin/cellulose microbead can be formed from a nanoemulsion prepared from a volatile oil and a non-volatile surfactant system.

A 900 nm nanoemulsion was prepared as follows: 0.021 g Montanov™ 82 (SEPPIC) was dissolved in 470 ml distilled water at 60 C. 10 g pinene was then poured into Montanov™ 82 solution and stirred at 60 C for 10 min at 1000 rpm. The mixture was then sonicated at 60% amplitude (Sonics® Vibra-Cell) in iced water bath for 20 min to produce an emulsion with an average droplet diameter of 900 nm.

300 mL of CNC suspension #1 (1.90 wt %) were poured into the above emulsion and mixed at 300 rpm for 10 min. 23 ml of fibroin solution #1 (1.88 wt %) were poured into the above mixture and stirred at 300 rpm for 10 min before spray-drying. The spray drier parameters were set as follows: inlet temperature 210 C, outlet temperature: 85 C, nozzle pressure 1.50 bar, differential pressure 180 mmWc, and nozzle air cap 70. The process yielded a dried free-flowing white powder.

The powder did not mix well with water and stayed on the water surface when added to water, indicating that the microparticles were hydrophobic.

Oil uptake was measured using the fluid saturation method as described in US standard ASTM D281-84. The oil uptake was measured to be 105 ml/100 g.

### Example 6—Porous Hydrophilic Fibroin/Cellulose Microparticles

When compared with Example 4, this Example, shows that the ratio of nanoemulsion to surfactant concentration affects whether the porous microbeads are hydrophobic or hydrophilic.

An 840 nm nanoemulsion was prepared as follows: 0.500 g Montanov™ 82 (SEPPIC) was dissolved in 350 ml distilled water at 60 C. 20 g pinene was then poured into Montanov™ 82 solution and stirred at 60 C for 15 min at 1000 rpm. The mixture was then sonicated at 60% amplitude (Sonics® Vibra-Cell) in iced water bath for 15 min to produce emulsions with an average droplet diameter of 840 nm.

466 mL CNC suspension #1 (2.16 wt %) were poured into the above emulsion and mixed at 300 rpm for 10 min. 12.7 ml of fibroin solution #1 (1.59 wt %) were poured into the above mixture and stirred at 300 rpm for 10 min before spray-drying. The spray drier parameters were set as follows: inlet temperature 210° C., outlet temperature: 85° C., nozzle pressure 1.50 bar, differential pressure 180 mmWc, and nozzle air cap 70. The process yielded a dried free-flowing white powder.

The powder sank quickly to the bottom of water once added to water, indicating that the microparticles were hydrophilic.

Oil uptake was measured using the fluid saturation method as described in US standard ASTM D281-84. The oil uptake was measured to be 185 ml/100 g.

### Example 7—Porous Hydrophilic Fibroin/Cellulose Microparticles

Compared with Example 4, this Example shows that a surfactant alone, interacting with the CNC, yields hydrophilic microparticles.

In this example, compared to Example 4, rather than using an emulsion comprising pinene/Montanov™ 82, a simple Montanov™ 82 solution was used.

440 mL CNC suspension #1 (2.16 wt %) was diluted with distilled water to 550 mL. 10 ml of fibroin solution #1 (1.99 wt %) was poured into the above suspension and stirred at 300 rpm for 10 min.

0.02 g MONTANOV™ 82 (SEPPIC) was dissolved in 50 mL distilled water. The MONTANOV™ 82 solution was added to the above mixture and stirred at 300 rpm for 3 min before spray-drying. The spray drier parameters were set as follows: inlet temperature 185 C, outlet temperature: 85 C, nozzle pressure 1.50 bar, differential pressure 180 mmWc, nozzle air cap 70. The process yielded a dried free-flowing white powder.

The powder mixed well with water when added to water, indicating that the microparticles were hydrophilic.

### Example 8—Sericin/Cellulose Microparticles

This Example shows that the protein Sericin can be incorporated into the cellulose microbead.

0.0051 g of sericin was dissolved in distilled water (2.3 mL), stirring at 500 rpm using a magnetic stir bar until no powder was visible and all appeared to be dissolved. The solution was filtered through a syringe filter 0.2 μm pore size and added to 17 mL of stirred CNC suspension #1 (3 wt %). The resulting mixture was sonicated (Sonics® Vibra-Cell) at 50% amplitude for 5 min while stirring. The suspension was spray-dried on a Buchi® B191 spray dryer using an inlet temperature of 175° C., aspirator 70%, pump speed 30%, air flow 600 Nl/h. The product was a free-flowing white powder that contained 1 wt % of sericin.

The following Table shows that other ranges of Sericin/Cellulose Microparticles can be obtained:

| Mass | Volume | Volume 3 wt | Final Sericin |
|---|---|---|---|
| Sericin (g) | water (mL) | % CNC (mL) | content (wt %) |
| 0.0024 | 2 | 17 | 0.5 |
| 0.0051 | 2 | 17 | 1 |
| 0.051 | 2 | 17 | 10 |
| 0.306 | 2 | 17 | 30 |

Compared to microparticles without sericin (such as those Comparative Example 9), the microparticles with sericin had a better skin feel, namely they felt creamier.

### Comparative Example 9—Cellulose Microparticles without Protein

This Example shows that cellulose microparticles prepared from CNC according to the method of International patent publication no. WO 2016\015148 A1, when spray dried to form microbeads, yield microbeads that are hydrophilic.

More specifically, CNC suspension #1 (4 wt % CNC) without any added protein was spray dried. The spray drier parameters were set as follows: inlet temperature 165-185° C., pump speed 30%, aspirator 70%, air pressure 600N1/h.

FIG. 2B) shows a sample of the powder obtained to which water was added. It can clearly be seen that the powder was wetted. This indicates that the microparticles are hydrophilic.

FIG. 3B) shows the powder obtained mixed the above water-in-oil emulsion. Aggregates were observed, indicating that the microparticles are hydrophilic.

### Comparative Example 10—Hydrophilic Porous Cellulose Microparticles without Protein

This Example shows that porous cellulose microbeads without added protein and are hydrophilic when prepared by a nanoemulsion method.

First, sodium carboxylate nanocrystalline cellulose (cCNC) was produced as described in International patent publication no. WO 2016 1015148 A1. As produced from the reaction of 30% aqueous hydrogen peroxide with dissolving pulp, a concentrated stock suspension of sodium carboxylate nanocrystalline cellulose (cCNC) consisted of 4% CNC in distilled water.

This stock suspension was diluted with distilled water. Then a poly(diallyldimethylammonium chloride) solution was prepared by diluting a 20 wt % solution of PDDA (Mw=400,000 to 500,000) with distilled water to prepare a solution of 2 wt %. The 4% sodium carboxylate CNC suspension was diluted to 1 wt %. Then, the 2 wt % PDDA solution was added to the carboxylate salt of CNC (cCNC) suspension at a solid mass ratio of 14% (PDDA/cCNC). The mixture was stirred for 3 min at 1000 rpm before sonication using flow cell with an amplitude of 60%, flow cell pressure of 20-25 psi, stirring rate of 1000 rpm. The resulting cationic cCNC+suspension was purified by diafiltration (Diafiltration unit (Spectrum Labs, KrosFlo TFF System)).

To prepare a nanoemulsion, 52.5 mL PEG-25 hydrogenated castor oil (Croduret™ 25), 52.5 mL Tween 80 (Polysorbate 80-Lotioncrafter), and 140 mL alkyl benzoate (C12-C15 Alkyl Benzoate, Lotioncrafter Ester AB) were poured into a 3.5 L glass beaker. Distilled water was added to the mixture to make the final volume 3.5 L. The mixture was stirred at 700 rpm for 20 min (VMI Rayneri Turbotest mixer). The mixture was then sonicated for 1.0 h at 60% amplitude (Sonics Vibra Cell) cooled in water bath to produce a nanoemulsion. After sonication, the nanoemulsion size was measured to be 45-50 nm by dynamic light scattering (NanoBrook 90 Plus, Brookhaven Instruments).

Then 0.84 wt % cCNC+ and 4.53 wt % CNC suspensions were prepared from the above stock suspensions. 2.8 L of the nanoemulsion was added to 3.9 L cCNC+(0.84 wt %) suspension with mixing at 400 rpm. After 5 min, 1.4 L cCNC (4.53 wt %) suspension were added and the mixture was stirred for another 5 min before spray-drying (parameters: inlet temperature 185 C, outlet temperature: 85 C, feed stroke 28%, nozzle pressure 1.50 bar, differential pressure 180 mmWc, nozzle air cap 70).

The process yielded a dried free-flowing white powder.

The sample was hydrophilic and exhibited a water uptake of 236 mL/100 g powder. The castor oil uptake was 252 mL/100 g of powder.

The scope of the claims should not be limited by the preferred embodiments set forth in the examples, but should be given the broadest interpretation consistent with the description as a whole.

### REFERENCES

The present description refers to a number of documents, the content of which is herein incorporated by reference in their entirety. These documents include, but are not limited to, the following:
International patent publication no. WO 2011/072365 A1;
International patent publication no. WO 2013/000074 A1;
International patent publication no. WO 2016/015148 A1;
International patent publication no. WO 2017/091893 A1;
Habibi et al. 2010, Chemical Reviews, 110, 3479-3500;
Y. Baimarck et al., “Morphology and thermal stability of silk fibroin/starch blended microparticles”, Polymer Letters Vol. 4, No. 12 (2010) 781-789; DOI: 10.3144/expresspolymlett.2010.94
Xie et al in “A Fully Biobased Encapsulant Constructed of Soy Protein and Cellulose Nanocrystals for Flexible Electromechanical Sensing”, DOI: 10.1021/acssuschemeng.7b0126, ACS Sustainable Chem. Eng. 2017, 5, 7063-7070
Sales et al. “Adsorption of Glycinin and 3-Conglycinin on Silica and Cellulose Surface Interactions as a Function of Denaturation, pH, and Electrolytes” (dx.doi.org/10.1021/bm2014153; Biomacromolecules 2012, 13, 387-396
Wu et al., “Nanocellulose/Gelatin Composite Cryogels for Controlled Drug Release” (ACS Sustainable Chem. Eng. 2019, 7, 6381-6389; DOI: 10.1021/acssuschemeng.9b0016)
J. S. Alves et al. “Effect of cellulose nanocrystals and gelatin in corn starch plasticized films”, Carbohydrate Polymers 115 (2015) 215-222; http://dx.doi.org/10.1016/j.carbpol.2014.08.057
H. Kwok et al., Facile and green fabrication of silk sericin films reinforced with bamboo-derived cellulose nanofibrils, Journal of Cleaner Production 200 (2018) 1034e1042; https://doi.oro/10.1016/j.jclepro.2018.07.289
Shang et al., “Intermolecular interactions between natural polysaccharides and silk fibroin protein”, Carbohydrate Polymers 93 (2013) 561-573; http://dx.doi.org/10.1016/j.carbpol.2012.12.038
Feng et al. “Facile Preparation of Biocompatible Silk Fibroin/Cellulose Nanocomposite Films with High Mechanical Performance; DOI: 10.1021/acssuschemeng.7b01161; ACS Sustainable Chem. Eng. 2017, 5, 6227-6236
Xiong et al., “Template-Guided Assembly of Silk Fibroin on Cellulose Nanofibers for Robust Nanostructures with Ultrafast Water Transport” (DOI: 10.1021/acsnano.7b04235, ACS Nano 2017, 11, 12008-12019
Van de Waterbeem, et al. Pure Appl. Chem. 1997, 69, 1137-1152
Chuang et al., Calculation of Contact Angle for Hydrophobic Powders Using Heat of Immersion Data, J. Phys. Chem. 1996, 100, 6626-6630).
Dong, X.; Revol, J.-F.; Gray, D., Effect of microcrystallite preparation conditions on the formation of colloid crystals of cellulose. Cellulose 1998, 5 (1), 19-32.
I. Pezron et al., Contact Angle Assessment of Hydrophobic Silica Nanoparticles Related to the Mechanisms of Dry Water Formation, Langmuir 2010, 26(4), 2333-2338; DOI: 10.1021/1a902759s
IUPAC. Compendium of Chemical Terminology, 2nd ed. (the “Gold Book”). Compiled by A. D. McNaught and A. Wilkinson. Blackwell Scientific Publications, Oxford (1997). XML on-line corrected version: http://goldbook.iupac.org (2006-) created by M. Nic, J. Jirat, B. Kosata; updates compiled by A. Jenkins. ISBN 0-9678550-9-8. https://doi.org/10.1351/goldbook.

1. Proteinaceous cellulose microparticles comprising cellulose nanocrystals and one or more peptide, one or more protein, or a mixture thereof, wherein the nanocrystals and the peptide(s) and/or protein(s) are aggregated together to form the microparticles.

2. The microparticles of claim 1, wherein the microparticles are from about 1 μm to about 100 μm in diameter.

3. The microparticles of claim 1, wherein the microparticles have a size distribution (D₁₀/D₉₀) of about 5/15 μm to about 5/25 μm by volume.

4. (canceled)

5. (canceled)

6. The microparticles of claim 1, wherein the cellulose nanocrystals have a crystallinity of at least about 50%.

7. The microparticles of claim 1, wherein the cellulose nanocrystals are sulfated cellulose nanocrystals and or a salt thereof, carboxylated cellulose nanocrystals or a salt thereof, or one of their derivatives, or a combination thereof.

8. The microparticles of claim 1, wherein the cellulose nanocrystals are carboxylated cellulose nanocrystals or a salt thereof.

9. The microparticles of claim 1, wherein the peptide and the protein are water-soluble.

10. The microparticles of claim 1, wherein the microparticles of the invention comprise one or more protein.

11. The microparticles of claim 1, wherein the microparticles comprise silk fibroin, sericin, or gelatin.

12. The microparticles of claim 11, comprising silk fibroin.

13. The microparticles of claim 1, being hydrophobic and lipophilic.

14. The microparticles of claim 1, wherein the microparticles comprise the one or more peptide and/or the one or more protein in a total peptide and protein concentration of about 0.1 wt % to about 50 wt %.

15. The microparticles of claim 1, wherein the microparticles are porous and the nanocrystals and the peptide and/or protein are arranged around cavities in the microparticles, thus defining pores in the microparticles.

16. The microparticles of claim 1, wherein the pores in the microparticles are from about 10 nm to about 2000 nm in size.

17. (canceled)

18. The microparticles of claim 1, wherein the cellulose nanocrystals are coated with a polyelectrolyte layer and a dye.

19. A cosmetic preparation comprising the microparticles of claim 1.

20. The cosmetic preparation of claim 19, comprising a water-in-oil emulsion or a lipophilic medium.

21. A method for producing the microparticles of claim 1, the method comprising the steps of:
a) providing a suspension of cellulose nanocrystals and a solution of the one or more peptide, one or more protein, or mixture thereof;
b) mixing the suspension with the solution to produce a mixture; and
c) spray-drying the mixture to produce the microparticles.

22. The method of claim 21, wherein the solution contains the one or more peptide, the one or more protein, or the mixture thereof in a concentration from about 0.01 wt % to about 50 wt % based on the total weight of the solution.

23. (canceled)

24. (canceled)

25. A method for producing the microparticles of claim 1 that are porous, the method comprising the steps of:
a) providing:
a suspension of cellulose nanocrystals,
a solution of the one or more peptide, one or more protein, or mixture thereof, and
an emulsion of a porogen,
wherein the solution of the one or more peptide, one or more protein, or mixture thereof either is part of the emulsion or stands alone;
b) mixing the suspension with the solution and the emulsion to produce a mixture comprising a continuous liquid phase in which:
droplets of the porogen are dispersed,
the cellulose nanocrystals are suspended and
the one or more peptide, one or more protein, or mixture thereof is dissolved;
c) spray-drying the mixture to produce microparticles; and
d) if the porogen has not sufficiently evaporated during spray-drying to form pores in the microparticles, evaporating the porogen or leaching the porogen out of the microparticles.
